(19) United States
(12) Patent Application Publication
(10) Pub. No.: US 2021/0315792 A1
(43) Pub. Date: Oct. 14, 2021

(54) DERMATOLOGICAL COMPOSITIONS FOR PROVIDING NUTRIENTS TO SKIN AND METHODS THEREOF

(71) Applicant: SOMAHLUTION, LLC, JUPITER, FL (US)

(72) Inventor: CATHERINE JULIE PACHUK, JUPITER, FL (US)

(73) Assignee: SOMAHLUTION, LLC, JUPITER, FL (US)

(21) Appl. No.: 17/261,929

(22) PCT Filed: Oct. 19, 2017
(86) PCT No.: PCT/US17/57463

§ 371 Date: Jan. 21, 2021

Related U.S. Application Data
(60) Provisional application No. 62/496,502, filed on Oct. 18, 2016.

(51) Int. Cl. A61K 8/64; A61K 8/67; A61K 8/20; A61K 8/19; A61Q 19/08; A61Q 19/00

(52) U.S. Cl. CPC A61K 8/64; A61K 8/676; A61K 8/20; A61K 8/19; A61K 2800/522; A61Q 19/007; A61K 2800/5922; A61K 2800/524; A61Q 19/08

(57) ABSTRACT

Disclosed are dermatological or cosmetic cream, serum and toner compositions and methods thereof for use in promoting healthy skin. A dermatological composition (cream, toner and serum) is applied to the skin to promote optimal health of the skin. The preferred cream composition is comprised of, reduced glutathione and/or cysteinylglycine, ascorbyl glucoside in a dermatologically acceptable carrier containing a salt composition. Preferably, the salt composition contains sodium, magnesium, potassium and calcium. The cream formulations are optionally ionically balanced. Preferably, potassium chloride, magnesium chloride, calcium chloride, magnesium sulfate and potassium phosphate are present in sufficient amounts. Toner composition contains sodium, magnesium, potassium and calcium ions and optionally bicarbonate ions and preservative(s). The toner formulations are optionally ionically balanced. The preferred serum composition contains ascorbyl glucoside and/or ascorbic acid in a carrier. Methods of manufacture of these compositions are also disclosed.

This application claims the benefit of U.S. Provisional Application No. 62/496,502 filed Oct. 18, 2016 and the text of application 62/496,502 is incorporated by reference in its entirety herewith.

### FIELD OF THE INVENTION

The present invention relates generally to dermatological cosmetic compositions that can be used on all types skin whether normal skin, dry skin, oily skin, or combination skin. In particular embodiments, the compositions are cream, serum and toner formulations.

### BACKGROUND OF THE INVENTION

As a mammal, especially a human, ages the skin of the mammal begins to deteriorate and form such things as wrinkles, crow's feet, and bags under the eyes. A wrinkle is a fold, ridge or crease in the skin. Skin wrinkles typically appear as a result of aging processes such as glycation or, temporarily, as the result of prolonged (more than a few minutes) immersion in water. Wrinkling in the skin is caused by habitual facial expressions, aging, sun damage, smoking, poor hydration, and various other factors. With prolonged water exposure, the outer layer of skin starts to absorb water. The skin doesn't expand evenly, causing it to wrinkle. Depletion of water in the body, as occurs with dehydration, can also cause this puckering of the skin. Hormones such as cortisol cause degradation of skin collagen.

Periorbital puffiness, also known as “puffy eyes” or swelling around the eyes, refers to the appearance of swelling in the tissues around the eyes, called the orbits. It is almost exclusively caused by fluid buildup around the eyes, or periorbital edema. Minor puffiness usually detectable below the eyes only is often called eye bags. While some degree of puffiness may be normal for a given individual, factors such as age and fatigue may make the swelling more prominent. The periorbital tissues are most noticeably swollen immediately after waking, perhaps due to the gravitational redistribution of fluid in the horizontal position. Eye puffiness may also be caused by: (a) Mononucleosis—With supra-orbital edema, the eyes become puffy and swollen. This may occur in the early stages of infection; (b) Oversleeping/sleep deprivation—Interrupted sleep cycles are common causes of eye puffiness; (c) Fluid retention—Many conditions (including pregnancy and hormonal variations with menstruation) can lead to the retention of fluid, particularly in the subcutaneous tissues. These conditions can cause swelling around the eyes to be more prominent; (d) Diet—Excess salt encourages fluid retention and may lead to puffy eyes; (e) Alcohol and tobacco use—Alcohol and tobacco contain toxins that may lead to stress, fatigue, and hormonal changes; all of which may lead to fluid retention and swelling around the eyes; (f) Allergies: Allergic reactions can lead to leaks in the subcutaneous capillary beds which can cause swelling in the face, including around the eyes; (g) Skin disorders—Eye puffiness can be a side effect of certain skin disorders, such as dermatitis, if the affected area becomes very sensitive, leading to swelling; (h) Normal aging—As a person grows older, the skin around the eyes becomes thinner and may swell or droop; (i) Crying: The salt in tears may cause fluid retention in the eye area; (j) Hypothyroidism—Facial puffiness and periorbital swelling occur due to infiltration with the mucopolysaccharides hyaluronic acid and chondroitin sulfate, pulling fluid into the interstitial space by osmosis; (k) Periorbital cellulitis—An inflammation and infection of the eyelid and portions of skin around the eye; (l) Tear glands—Puffiness around the eyes can also be due to the improper functioning of the tear glands.

Aging of the skin can be classified into two components: intrinsic and extrinsic aging. As the names imply, intrinsic aging is due to genetically controlled senescence and extrinsic aging is due to environmental factors superimposed on intrinsic aging. Environmental factors known to accelerate extrinsic aging are sun exposure and cigarette smoking. Cutaneous aging of skin due to sun exposure is known as photoaging.

Skin includes three layers: epidermis, dermis and subcutaneous tissue. Among these, dermis contains a high amount of collagen and elastin which are extracellular matrix components. The components are important for maintenance of skin functions such as skin elasticity and water retentivity. Youthful skin is characterized by its unblemished, evenly pigmented, smooth, pink and firm appearance. This is in contrast to intrinsically aged skin (senile change of skin), which is thin, inelastic and finely wrinkled with deepening of facial expression lines. These changes are evident histologically as a thinned epidermis and dermis with flattening of the rete pegs at the dermoepidermal junction. Extrinsically aged, sun-exposed skin appears clinically as blemished, thickened, yellowed, lax, rough, leathery and with loss of luminosity. These changes may begin as early as the second decade. Photoaged skin is characterized histologically by epidermal dysplasia with varying degrees of cytologic atypia, loss of keratinocyte polarity, an inflammatory infiltrate, decreased collagen, increased ground substance and elastosis. Elastosis is the degradation of elastic material, which, in early photoaging, is increased in amount and seen microscopically as thickened, twisted, degraded elastic fibers that result in a loss of skin elasticity and an increase in fine lines and wrinkles and loss of skin firmness.

The ability of collagen and elastin production of fibroblasts and the migration ability of keratinocytes from the epidermal basal layer to stratum corneum are also important for the wound healing. For example, when dermis is lost due to a sever wound, granulation tissue must be generated to fill the region of the wound. The granulation tissue includes extracellular matrix such as fibroblasts and collagen produced by fibroblasts. Further, epidermis lost due to the wound is repaired by keratinocytes which migrate from the epidermal basal layer surrounding the region of the wound to cover the region of the wound.

It is generally known that the deficiency of the appropriate skin care may lead to various skin problems, which may include accelerated skin aging, skin disorders and diseases. There are a myriad of skin care products available to consumers. Further, there are different skin types among human population. Such skin types range from normal skin, dry skin, oily skin, and combination skin (e.g., normal/dry, normal/oily, dry/oily). This leads to a confusing and exhaustive search for different products for different applications (cleanser, toner, serum, moisturizing cream, etc.).

For the purpose of improvement of skin or prevention of senile change of skin various compositions have been developed so far including compositions containing extracellular matrix components such as collagen and compositions containing saccharides, amino acids, organic acids and pyrrolidone carboxylic acid. These aim, for aesthetic purposes, to: (i) prevent or delay the appearance of the signs of extrinsic and/or intrinsic ageing of the skin. (ii) improve the uniformity of the skin color, or (iii) to correct or reduce pigmentary spots on the skin. In this regard, a number of cosmetic moisturizing creams, serums and toners are known and used in the marketplace but are not quite satisfactory. For instance, toners include high levels of acetone or alcohol (e.g., at least 20 to 70% w/w) such ethanol, acetone, or isopropanol. These alcoholic-based toners can be caustic or irritating to skin. Other toners also use high levels (e.g., at least 20 to 70% w/w) of glycol-based ingredients (e.g., glycol ethers), which can be sharp or biting to the taste or smell and irritating to the eyes, nose, etc. While some water-based toners containing high amounts of surfactants or emulsifiers exist, the use of surfactants or emulsifiers can irritate the skin and high amounts of water can preclude the addition of other ingredients beneficial to skin.

The search for new dermatological compositions—creams, moisturizing creams, serums and toners—which are effective and devoid of toxicity is therefore a necessity in the cosmetic industry. Thus, there is a need to develop a dermatological formulation to provide nutrition to the skin to help prevent wrinkles, puffiness under the eyes, dry skin and wind-burned skin.

### SUMMARY OF THE INVENTION

The present invention fills this need by providing for compositions and methods for promoting healthy skin involving nutrient-rich and/or antioxidant-filled cream, serum and toner formulations. Cream, moisturizing cream, serum and toner disclosed herein are all topical skin care products. The main purpose of the invention is to solve the technical problem involving the provision of novel dermatological or cosmetic compositions containing select ingredients, the compositions being intended for preventing or delaying the appearance of the signs of extrinsic and/or intrinsic ageing of the skin, or for slowing down or reducing the effects thereof at least in areas the cosmetic composition is applied to.

In an aspect of the invention, a dermatological cream composition containing reduced glutathione or cysteinylglycine or a combination of reduced glutathione and cysteinylglycine, and at least one antioxidant in a dermatologically acceptable carrier is provided. The antioxidant is ascorbyl-2-glucoside (AA-2G) and/or ascorbyl-2-glucosamine. In a preferred embodiment, a dermatological cream composition contains reduced glutathione or cysteinylglycine or a combination of reduced glutathione and cysteinylglycine, and at least one of ascorbyl-2-glucoside (AA-2G), ascorbyl-2-glucosamine and ascorbic acid in a dermatologically acceptable carrier. In another preferred embodiment the dermatological cream composition contains electrolytes in an effective amount of each of Na⁺, K⁺, Ca⁺, Mg²⁺, and Cl⁻, and optionally HCO₃⁻, more preferably in amounts sufficient for achieving ionic balance. In one preferred embodiment of the invention, the dermatological cream composition contains at least one antioxidant that is ascorbyl-2-glucoside (AA-2G) or ascorbyl-2-glucosamine in combination with reduced glutathione and the composition is ionically balanced.

In another embodiment, the dermatological cream composition contains additional antioxidants at least one of which is selected from the group consisting of: carnosine, resveratrol, ascorbic acid, ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate and quercetin. The dermatological cream composition further contains L-arginine and/or sugar such as glucose. In general aspects of this invention, not just limited to this embodiment, where glucose is used, any other sugar can be used in place of or in addition to glucose but preferably a monosaccharide including but not limited to fructose, mannose and ribose is used.

In yet another embodiment, the dermatological cream composition contains glucose, arginine, reduced glutathione or cysteinylglycine or a combination of reduced glutathione and cysteinylglycine, and at least one of ascorbyl glucoside, ascorbyl-2-glucosamine and ascorbic acid in a dermatologically acceptable carrier. This composition is preferably ionically balanced. In can contain one or more additional antioxidants selected from the group carnosine, resveratrol, ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate and quercetin.

In another embodiment, the dermatological cream composition contains reduced glutathione or cysteinylglycine or a combination of reduced glutathione and cysteinylglycine, and at least one of ascorbyl glucoside, ascorbyl-2-glucosamine and ascorbic acid, in a dermatologically acceptable carrier with the proviso that the composition does not contain a pigment. It can further contain arginine and a sugar.

In another embodiment, a method for promoting healthy skin is provided. The method involves applying to the skin a dermatological cream composition containing reduced glutathione or cysteinylglycine or a combination of reduced glutathione and cysteinylglycine, and an antioxidant (ascorbyl-2-glucoside or ascorbyl-2-glucosamine) in a dermatologically acceptable carrier. Preferably, the dermatological cream composition is ionically balanced. The dermatological cream composition can contain ascorbic acid in addition to ascorbyl-2-glucoside or ascorbyl-2-glucosamine or in place of ascorbyl-2-glucoside and/or ascorbyl-2-glucosamine, and optionally contains ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate or quercetin, or a combination these antioxidants.

In yet another embodiment, another method for promoting healthy skin is provided. The method involves applying to the skin a dermatological cream composition containing an effective amount of sodium, magnesium, potassium, calcium and chloride ions, optionally HCO₃⁻, and a sugar, arginine, reduced glutathione and ascorbyl glucoside or ascorbyl-2-glucosamine in a dermatologically acceptable carrier. Optionally, the dermatological cream composition is ionically balanced.

In another aspect of the invention, a dermatological toner composition containing a significant portion of water (at least 85% by weight of water based on total weight of the composition), and sodium, magnesium, potassium, calcium and chloride ions, and optionally HCO₃⁻ is provided. These electrolytes are present in an effective amount so the toner is effective. The dermatological toner composition is preferably ionically balanced. It may contain preservatives or a preservative system free of parabens, formaldehyde and isothiazolinones. It contains caprylyl glycol, phenoxyethanol or propylene glycol, or ethylhexylglycerin. Preferably, pH of the toner composition is about 6.0 and density is about 1.0 g/ml. A method for promoting healthy skin is also provided. It involves applying to the skin any dermatological toner composition in sufficient amount for promoting healthy skin.

In yet another aspect of the invention, a dermatological serum composition containing reduced glutathione or cysteinylglycine or a combination of reduced glutathione and cysteinylglycine, and at least one antioxidant in a dermatologically acceptable carrier. The antioxidant can be ascorbyl-2-glucoside (AA-2G) and/or ascorbyl-2-glucosamine). In one preferred embodiment, the composition contains additional antioxidants in addition to or not inclusive of AA-2G and/or ascorbyl-2-glucosamine (e.g., ascorbic acid, carnosine, resveratrol and ascorbyl palmitate). In an embodiment of the invention, the dermatologically acceptable carrier for serum compositions is water, propanediol, sodium hydroxide, phenoxyethanol and ethylhexylglycerin and optionally sodium hyaluronate. This serum composition can further contain xanthan gum or hydroxyethylcellulose, and optionally citric acid.

In another embodiment, the dermatologically acceptable carrier for serum compositions is water, propanediol, sodium hydroxide, phenoxyethanol and ethylhexylglycerin and optionally sodium hyaluronate, SD alcohol 40-B, bis-PEG-12 dimethicone and xanthan gum.

In yet another embodiment, the dermatologically acceptable carrier for serum compositions is water, propanediol, sodium hydroxide, phenoxyethanol and ethylhexylglycerin (sodium hyaluronate, optional), SD alcohol 40-B, bis-PEG-12 dimethicone and xanthan gum. SD alcohol 40-B and bis-PEG-12 dimethicone (and citric acid, optional).

In yet another embodiment, a dermatological serum composition contains a dermatologically acceptable carrier containing reduced glutathione, ascorbyl glucoside and ascorbic acid. The dermatologically acceptable carrier contains water, propanediol, sodium hydroxide, phenoxyethanol and ethylhexylglycerin and optionally at least one of xanthan gum hydroxyethylcellulose and sodium hyaluronate. The dermatological serum composition can further contain SD alcohol 40-B and bis-PEG-12 dimethicone (and citric acid, optional).

In yet another embodiment, a method for promoting healthy skin is provided. It involves applying to the skin a dermatological serum composition containing reduced glutathione or cysteinylglycine, or a combination of reduced glutathione and cysteinylglycine, and ascorbyl glucoside and ascorbic acid, and optionally citric acid, in a dermatologically acceptable carrier. The dermatologically acceptable carrier contains water, propanediol, sodium hydroxide, phenoxyethanol and ethylhexylglycerin and optionally xanthan gum or hydroxyethylcellulose and sodium hyaluronate. It can further contain SD alcohol 40-B and bis-PEG-12 dimethicone.

### BRIEF DESCRIPTION OF THE DRAWINGS

FIG. 1 is an exemplary cream composition of the invention.

FIG. 2 is another exemplary cream composition of the invention.

FIG. 3 is another exemplary cream composition of the invention.

FIG. 4 is another exemplary cream composition (with 0.25% AA2G) of the invention.

FIG. 5 is another exemplary cream composition (1942604 batch A) of the invention.

FIG. 6 is another exemplary cream composition (1942604 batch B) of the invention.

FIG. 7 is another exemplary cream composition (with 0.1% ascorbic acid) of the invention.

FIG. 8 is another exemplary cream composition (with 0.01% ascorbic acid) (1942611) of the invention.

FIG. 9 is another exemplary cream composition (without AA2G and glutathione) (1942609) of the invention.

FIG. 10 is another exemplary cream composition (with 10% AA2G) of the invention.

FIG. 11 is another exemplary cream composition (with 0.001% ascorbic acid) of the invention.

FIG. 12 is another exemplary cream composition of the invention.

FIG. 13 is a chart showing stability testing data of various dermatological cream compositions of the invention.

### DETAILED DESCRIPTION OF THE INVENTION

This invention concerns cosmetic or dermatological compositions in particular moisturizing creams, serums and toners and methods of use of same for: i) preventing or delaying the appearance of the signs of extrinsic and/or intrinsic aging of the skin, or ii) reducing the effects thereof, at least in areas the cosmetic composition is applied to.

In general, the compositions contain water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties. These same general ingredients can be formulated into a cream rather than a lotion, or into gels, or into solid sticks by utilization of different proportions of the ingredients and/or by inclusion of thickening agents such as gums or other forms of hydrophillic colloids. Such compositions are referred to herein as “dermatologically acceptable carriers” unless otherwise specifically provided herein. Most preferred for skin are those carriers that are fat-soluble, i.e., those which can effectively penetrate skin layers and deliver nutrients to the lipid-rich layers of the skin. In the compositions herein that contain glucose, any other sugar can be used in place of or in addition to glucose such as fructose, mannose or ribose.

In one aspect of the invention, the dermatological composition is a cream formulation (moisturizing or otherwise). The cream formulation contains, in a dermatologically acceptable carrier, reduced glutathione, optionally cysteinylglycine, and an antioxidant.

In a preferred embodiment, the antioxidant substance is an ascorbyl compound that has a moiety attached thereto that inhibits oxidation of the ascorbyl compound. In a preferred embodiment the ascorbyl compound is ascorbyl glucoside. In another embodiment, the antioxidant substance is ascorbic acid/L-ascorbic acid.

Additional antioxidants that inhibit degradation or oxidation of glutathione or that promote the stability of the reduced glutathione may be added to the present formulation including but not limited to carnosine, resveratrol, ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate and quercetin. Preferably the antioxidant is one that inhibits degradation or oxidation of glutathione or that promotes the stability of the reduced glutathione.

In addition to the above, arginine and a sugar, preferably glucose, can be added to the composition. In an embodiment, cysteinylglycine can be substituted for reduced glutathione.

In a preferred embodiment, the nutrient-rich antioxidant-filled dermatological composition of the present invention has reduced glutathione (optionally cysteinylglycine) ascorbyl glucoside, in a dermatologically acceptable carrier. In addition, L-arginine and a sugar can be present in the composition. Within the dermatologically acceptable carrier, ions or electrolytes—Na⁺, K⁺, Cl⁻, Ca²⁺, and Mg²⁺, and optionally HCO₃⁻ can be present. Various inorganic salts are added to the dermatologically acceptable carrier of cream or toner composition for these electrolytes. Examples of the source of those key ions are sodium chloride, potassium chloride, sodium bicarbonate, calcium chloride, magnesium chloride, magnesium sulfate, potassium phosphate, sodium phosphate. Such salts can be added specifically for the purpose of having ions or electrolytes Na⁺, K⁺, Cl⁻, Ca²⁺, and Mg²⁺, and optionally HCO₃⁻ in sufficient amounts. The salts can be added for achieving ionic balance. Other salts such as disodium EDTA may also account for the relevant ion (Na⁺ from disodium EDTA) for purposes of ionic balance.

TABLE 2
Ionic Balance

| | Normal Ionic | Concentration in a | Concentration in a |
|---|---|---|---|
| | Concentration Range | Cream Embodiment | Toner Embodiment |
| Ion | (mM) | (mM) | (mM) |
| Na⁺ | 135-147 | 91.8 | 137.5 |
| K⁺ | 3.5-5.1 | 10.0 | 5.82 |
| Ca²⁺ | 1.0-1.3 | 1.6 | 1.0 |
| mg²⁺ | 1.5-2.3 | 1.5 | 0.9 |
| Cl⁻ | 95-110 | 87.7 | 145.4 |

In the context of various compositions herein, the term “ionically balanced” means that a given composition must contain the following key anions and cations: Na⁺, K⁺, Ca²⁺Mg²⁺, and Cl⁻ at concentrations that are within 6 mM of the normal ionic concentration range for K, Ca and Mg ions and within 33% of the normal ionic concentration range specified for Na and Cl ions. In certain embodiments of the invention, cream and toner compositions are ionically balanced compositions. In certain other embodiments of the invention, cream or moisturizing cream and toner compositions are not ionically balanced compositions but contain a sufficient amount or an effective amount (more than mere trace amount) of each of Na⁺, K⁺, Cl⁻, Ca²⁺, and Mg²⁺, and optionally HCO₃⁻ whether or not ionically balanced. An example of the sufficient amount or concentration of these electrolytes is about 5.8 mM (K⁺), 156.6 mM (Na⁺), 145 mM (Cl⁻), 0.9 mM (Mg²⁺), 1.0 mM (Ca²⁺), and 5.8 mM (K⁺), and optionally 19.3 (HCO₃⁻). Ionically balanced compositions (with or without HCO₃⁻) disclosed herein are other examples for providing guidance to one skilled in the art as to the sufficient amount or effective amount of Na⁺, K⁺, Ca²⁺Mg²⁺, and Cl⁻, and optionally HCO₃⁻.

Generally topical application to exposed or affected skin sites is accomplished in association with a carrier, and particularly one in which the ingredients of the present invention are soluble or incorporated into an emulsion, in particular dermatologically acceptable carrier. In one embodiment, density (g/L) of cream composition is about 0.9, preferably about 0.97. While the carrier can be comprised of a relatively simple solvent or dispersant such as oils, and optionally salts for ionic balance, it is generally preferred that the carrier contains composition more conducive to topical application, and particularly one which will form a film or layer on the skin to which it is applied so as to localize the application and provide some resistance to perspiration and/or one which aids in percutaneous delivery and penetration of the active ingredients into lipid layers. An example of a dermatologically acceptable carrier that is more conducive to topical application has 1, 2, 3, 4, 5, 6, 7, 8, 9 or 10, or 11-34 or all 35 of the following ingredients: one or more ceramide ingredients (selected from the group consisting of: ceramide NP, ceramide NS, ceramide EOS, ceramide EOP, ceramide AP, caprooyl phytosphingosine, caprooyl sphingosine), jojoba esters, Salix nigra (willow) bark extract, cyclopentasiloxane, polysilicone-11, hydroxyethylacrylate/sodium acryloyldimethyl taurate copolymer, polyisobutene, peg-7 trimethylolpropane coconut ether, squalene, propanediol, cetearyl alcohol, ceteareth-20, butylene glycol, Morus alba root extract, glucose, polyacrylate crosspolymer-6, glyceryl stearate, peg-100 stearate, Butyrospermum parkii (shea butter) (and/or fatty acids such as cocoa butter, palm oil, coconut oil, soybean oil, rapeseed oil, cottonseed oil and Borneo tallow nut oil), glycerin, Camellia sinensis leaf extract, phenoxyethanol, ethylhexylglycerin, ceteareth-25, cetyl alcohol, behenic acid, hydrolyzed hyaluronic acid, disodium EDTA and tocopheryl acetate.

In an embodiment, the dermatological cream composition of the present invention has water, jojoba esters, Salix nigra (willow) bark extract, cyclopentasiloxane, polysilicone-11, hydroxyethylacrylate/sodium acryloyldimethyl taurate copolymer, polyisobutene, peg-7 trimethylolpropane coconut ether, squalene, propanediol, cetearyl alcohol, ceteareth-20, butylene glycol, Morus alba root extract, glucose, polyacrylate crosspolymer-6, glyceryl stearate, peg-100 stearate, Butyrospermum parkii (shea butter), glycerin, Camellia sinensis leaf extract, phenoxyethanol, ethylhexylglycerin, ceteareth-25, cetyl alcohol, behenic acid, at least one ceramide ingredient (ceramide NP, ceramide NS, ceramide EOS, ceramide EOP, ceramide AP, caprooyl phytosphingosine, and caprooyl sphingosine), hydrolyzed hyaluronic acid, arginine, glutathione, L-ascorbic acid and/or ascorbyl glucoside (AA-2G), disodium EDTA, tocopheryl acetate, sodium chloride, potassium chloride, sodium bicarbonate, calcium chloride, magnesium chloride, magnesium sulfate, potassium phosphate monobasic and sodium phosphate dibasic anhydrous.

In another aspect of the invention, the dermatological composition is a toner. By this invention, a solution to the problems associated with current cosmetic toners has been discovered. That solution is the use of a composition having, among other things, a combination of salts, as a toning formulation. The toning formulation is preferably ionically balanced. In one embodiment, density (g/L) of toner composition is about 1.000 preferably about 1.002.

In a broad aspect of the toner composition, there is disclosed topical toner formulations. The toner formulations include a combination of salts in at least 85% by weight of water (based on total weight of the composition). Toner formulations of the present invention are ionically balanced. An example of the combination of salts is: sodium chloride, potassium chloride, calcium chloride, magnesium chloride, magnesium sulfate, potassium phosphate monobasic, sodium phosphate dibasic anhydrous, and optionally sodium bicarbonate, as a source for the respective cations and anions in the toner formulation. In this regard, the amount of any one of the salts within a given composition can range from (by w/w) 0.1 to 1.0%, 0.01% to 0.05%, 0.005 to 0.05%, 0.0014 to 0.014%, 0.0009 to 0.006%, 0.002% to 0.0027%, 0.00001 to 10%, 0.0001 to 5%, 0.001 to 2%, 0.01 to 1%, 0.1 to 0.5%. 0.001-0.003%. It can be, for example, about 0.003% w/w, 0.006% w/w, 0.10% w/w, 0.014% w/w, 0.014% w/w, 0.5% w/w, 0.8% w/w, 1.0% w/w, 1.2% w/w or 1.5% w/w of the composition.

It is also contemplated, however, that in certain embodiments the amount of the salts can go below or above the stated concentrations (or concentration ranges) for blood plasma.

The toner formulation can serve as a topical cosmetic vehicle wherein the amount of water can be modified to account for preservatives and other ingredients and optionally one or more botanical extracts. The toner formulation contains high amounts of water and ions—K⁺, Na⁺, Cl⁻, Ca₂⁺, and Mg₂⁺. The anion HCO₃⁻ may or may not be present. In an embodiment of the invention, the cosmetic vehicle can also include at least one preservative system. The preservative system is preferably free of parabens, formaldehyde, and isothiazolinones. An example of a preservative system preferably free of parabens, formaldehyde, and isothiazolinones is caprylyl glycol, phenoxyethanol and propylene glycol (or ethylhexylglycerin). Any or all of caprylyl glycol, phenoxyethanol and propylene glycol can be used. The general range/amounts for each of these ingredients in the toner can be (based on total weight of the composition): 0.001% to 1.5% w/w. It can be, for example, about 0.003% w/w, 0.006% w/w, 0.10% w/w, 0.014% w/w, 0.014% w/w, 0.5% w/w, 0.8% w/w, 1.0% w/w, 1.2% w/w or 1.5% w/w of the composition. The source of electrolytes—K⁺, Na⁺, Cl⁻, Ca₂⁺, Mg₂⁺ and HCO₃⁻— is exemplified above.

In some embodiments, any combination of or at least 1, 2, 3, 4, 5, 6, 7, 8, 9, 10, 11, or all 12 of the following additional ingredients can be included in the composition: butylene glycol; glycerin; diazolidinyl urea; methylparaben; disodium EDTA; simethicone; PPG-26; PEG/PPG-22/23 dimethicone; citric acid; phenoxyethanol; potassium sorbate; and sodium benzoate. However, it is preferred but not necessary that the composition is free of parabens, formaldehyde, and isothiazolinones. The general range/amounts for each of these ingredients in the vehicle can be (based on total weight of the composition): 0.2 to 0.8% by weight of butylene glycol; 1 to 2% by weight of glycerin; 0.1 to 0.3% by weight of diazolidinyl urea; 0.1 to 0.2% by weight of methylparaben; 0.05 to 0.1% by weight of disodium EDTA; 0.002 to 0.003% by weight of simethicone; 0.001 to 0.002% by weight of PPG-26; 0.001 to 0.002% by weight of PEG/PPG-22/23 dimethicone; 0.0001 to 0.002% by weight of citric acid; 0.0001 to 0.0007% by weight of phenoxyethanol; 0.001 to 0.003% by weight of potassium sorbate; and 0.00001 to 0.0002% by weight of sodium benzoate.

The ionically balanced toner formulations with its high amounts of water, and key ions—K⁺, Na⁺, Cl⁻, Ca₂⁺, and Mg₂⁺, and optionally HCO₃⁻— in the cosmetic vehicle work well as toners across all skin types (e.g., normal skin, dry skin, sensitive skin, oily-skin, combination skin—e.g., normal/dry, normal/oily, dry/oily) to enhance skin's surface, giving skin what it needs to look fresher, smoother, and hydrated. Without wishing to be bound by any theory, it is believed that these formulations work well as toners across all skin types because these are ionically balanced toner formulations. While one may certainly use a toner alone and skip the serum described herein for addressing specific skincare concerns, one may realize benefits to using both This combination can be used across all skin types (e.g., dry skin, normal skin, oily skin, and combination skin).

In another aspect of the invention, the dermatological composition is a serum. Serum of the present invention is a topical skincare product. It can be applied topically to skin optionally after cleansing but before moisturizing for delivering powerful ingredients directly into the skin. Serum of the present invention is particularly suited to this task because it is composed of small molecules that can penetrate deeply into the skin and along the way deliver a very high concentration of ascorbic acid and ascorbyl glucoside, among others. The serum of the present invention serves as a ready tool for targeting/treating specific skincare concerns, like hyperpigmentation, fine lines, and wrinkles. It can also protect skin from UV damage.

In general, the serum contains an antioxidant in a dermatologically acceptable carrier. Such antioxidants have already been discussed herein (e.g., ascorbyl glucoside (AA-2G) or ascorbyl-2-glucosamine). In addition to ascorbyl glucoside (AA-2G) or ascorbyl-2-glucosamine, the formulation can contain ascorbic acid and/or reduced glutathione, optionally cysteinylglycine (or cysteinylglycine can be substituted for reduced glutathione). In an embodiment, the antioxidant-filled dermatological composition (cream or serum) of the present invention contains one or more of these specific antioxidants described above and does not contain ascorbyl-2-glucosamine and/or ascorbyl-2-glucoside. In one embodiment, density (g/L) of serum composition is about 1.09, preferably about 1.098.

An example of a dermatologically acceptable carrier for serum compositions is water, propanediol, sodium hydroxide, phenoxyethanol and ethylhexylglycerin. The carrier can contain 1, 2 or more or all of following other components: xanthan gum, sodium hyaluronate, hydroxyethylcellulose, SD alcohol 40-B and bis-PEG-12 dimethicone.

In an embodiment of the invention, the dermatological serum composition contains water, ascorbic acid, ascorbyl glucoside, propanediol, sodium hyaluronate, sodium hydroxide, phenoxyethanol, ethylhexylglycerin, Glutathione. Xanthan gum can be added to this formulation to improve stability at or above room temperature (e.g., 40° C.). In another embodiment of the invention, hydroxyethylcellulose in addition to xanthan gum or in place of xanthan gum is added to the serum formulation for improving stability at higher temperatures (e.g., 40° C.). The formulation can optionally contain citric acid.

In another embodiment of the invention, the serum composition is composed of water, ascorbic acid, ascorbyl glucoside, propanediol, sodium hyaluronate, sodium hydroxide, phenoxyethanol, ethylhexylglycerin, glutathione. Xanthan gum can be added to this formulation to improve stability at or above room temperature (e.g., 40° C.). In another embodiment of the invention, hydroxyethylcellulose in addition to xanthan gum or in place of xanthan gum is added to the serum formulation for improving stability at higher temperatures (e.g., 40° C.). The above serum embodiment containing hydroxyethylcellulose in addition to xanthan gum or in place of xanthan gum may further contain SD alcohol 40-B and Bis-PEG-12 Dimethicone. Applicant discovered that these additional components (singly or together) can help reduce tackiness. In one preferred embodiment, the above formulation containing SD alcohol 40-B and Bis-PEG-12 Dimethicone does not contain xanthan gum and/or hydroxyethylcellulose and hyaluronic acid/sodium hyaluronate. This may avoid formation of gel particles. Any of the serum compositions herein can optionally contain citric acid. The pH of the formulation is set to about 5.0-6.0 or physiological pH.

Method of making cream composition is disclosed. In an embodiment of the present invention, cream composition can be made by producing eight different mixtures each called a “phase” (e.g., phase 1 or A, phase 2 or B, phase 3 or C, phase 4 or D, phase 5 or E, phase 6 or F, phase 7 or G and phase 8 or H) and carrying out method steps. In an embodiment, the various phases and steps for making cream composition involve the following.

Phase 1 is prepared by dispersing polyacrylate crosspolymer-6 in water and heating it to between 65 and 70° C. and then Propanediol and/or disodium EDTA are added while heating and mixing. This completes step 1.

Phase 2 is prepared by mixing 2 or more of the components—selected from the group consisting of: hydroxyethylacrylate/sodium acryloyldimethyl taurate copolymer, polyisobutene, PEG-7 trimethylolpropane coconut ether, cyclopentasiloxane polysilicone-11, tocopheryl acetate, cyclopentasiloxane, glyceryl stearate, PEG-100 stearate, jojoba esters, Butyrospermum parkii shea butter, cetearyl alcohol ceteareth-20 and squalene—and warming it to between 65 and 70° C. The composition of Phase 2 is mixed together with the composition of Phase 1 and homogenized for 5-10 minutes by centrifugation at about 4000 rpm or until the mixture became uniform. The mixture is then cooled to 44.5-50° C., if needed, with mixing. This completes step 2.

Phase 3 is prepared by dissolving in water one or 2, 3, 4, 5, 6, 7 or all 8 of the components as source of some or all of the anions and cations Na⁺, K⁺, Cl⁻, Ca²⁺, Mg²⁺, and HCO₃⁻— the component selected from the group consisting of: sodium chloride, potassium chloride, magnesium chloride, calcium chloride, magnesium sulfate, potassium phosphate monobasic, sodium bicarbonate, sodium phosphate dibasic anhydrous—and the resultant solution is added to the composition produced by step 2 or vice versa. One skilled in the art knows that certain salts are available in anhydrous form as well as forms with 2, 7, 8, and 12 hydrates and any of these water soluble salts can be used adjusting amounts accordingly to make up the desired molar solutions. This completes step 3.

Phase 4 is prepared by dissolving hydrolyzed hyaluronic acid in water. This Phase 4 composition is added to the composition of step 3. This completes step 4.

Phase 5 is prepared by adding various components; one or more of the components selected from the group consisting of: ceteareth-25, glycerin, cetyl alcohol, and behenic acid; at least one ceramide ingredient (ceramide NP, ceramide NS, ceramide EOS, ceramide EOP, ceramide AP, caprooyl phytosphingosine, and caprooyl sphingosine); and any or all of: water and Salix nigra (willow) bark extract; butylene glycol and Morus alba root extract; water, glycerin and Camellia sinensis leaf extract. In addition, arginine, glutathione, and optionally glucose are added to prepare phase 5. This completes step 5. It is preferred that the components of phase 5 are added one at a time to the composition of step 4 and see that each component is thoroughly dissolved in the mixture before the next component one is added.

Phase 6 is phenoxyethanol ethylhexylglycerin. It is added to the composition of step 5 or vice versa and mixed until it is uniformly dispersed in the composition. This completes step 6.

Phase 7 is ascorbic acid (AA) or ascorbyl glucoside (AA-2G) or both AA and AA-2G. L-ascorbic acid can be added to phase 6 and phase 7 comprises AA-2G. Phase 7 is added to the composition of step 6 or vice versa and thoroughly mixed. This completes step 7.

Phase 8 is 20% solution of citric acid in water used as a buffering agent. It is added to the composition of Step 7 until a pH of 3.8 to 4.2 is obtained. This completes step 8.

In certain working examples herein, the term “batch” is used to refer to the composition or mixture of the prior step of the process. It should be noted that in some embodiments, the order of phases can be different. For example, phase 1 components in one embodiment can be phase 2 in another embodiment, phase 3 components in one embodiment can be phase 5 in another embodiment and so on. Likewise, one or more components from one phase in one embodiment can be added to another phase in another embodiment and so on. For example, ascorbic acid (AA) can be in phase 5, not in phase 7.

A method of making toner composition is also disclosed. An effective amount of inorganic salts, as a source of ions or electrolytes—Na⁺, K⁺, Cl⁻, Ca²⁺, and Mg²⁺— (e.g., sodium chloride, potassium chloride, magnesium chloride, calcium chloride, magnesium sulfate, potassium phosphate, and sodium phosphate—not listed in any predominant order) are slowly added into a beaker containing water, preferably purified water while mixing vigorously at about 250-1000 rpm using, for example, a 3″ propeller blade until homogeneous. The density of toner may be about 1 g/ml. The quantity of water (% w/w) should be at least 90%, preferably at least 95%. The pH of the resulting solution is adjusted to 6.0±0.25. An appropriate pH adjusting agent (e.g., either HCl or NaOH as needed) can be used. Preferably, the amount of each of the inorganic salts for the toner composition is sufficient enough for achieving ionic balance (See Table 2, Ionic Balance). In this manner, the toner can be ionically balanced. Osmolality (mOsm/kg) of the toner can be, for example, about 280, preferably about 288.

The compositions and methods of their use or manufacture can “comprise,” “consist essentially of,” or “consist of” any of the ingredients/components disclosed throughout the specification. For purposes of invention(s) herein, the term consisting essentially of means that the inclusion of additional ingredients in the compositions do not materially affect the properties of the aforementioned combination of ingredients/components in cream, toner and serum, and cosmetic vehicle. One such instance would be the inclusion of an ingredient that has a detrimental effect on (e.g., reducing the efficacy or stability of) any one of the ingredients identified said combination.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “about” “approximately” “of the order of” or “substantial(ly), a term of degree modifying the quantity of an ingredient in the compositions of the invention or employed in the methods of the invention refers to variation in the numerical quantity that can occur, as understood by one of ordinary skill in the art, for example, through typical measuring, weighing and/or solution handling procedures used for making concentrates or use compositions in the real world; through inadvertent error in these procedures; through differences in the manufacture, source, or purity of the ingredients employed to make the compositions or carry out the methods; and the like. Whether or not modified by the term “about” “approximately” “of the order of” or “substantial(ly), it is intended that the claims include equivalents to the quantities. In one non-limiting embodiment the terms are defined to be within 10%, preferably within 5%, more preferably within 1%, and most preferably within 0.5%.

The term “promoting” or any variation of this term, when used in the claims and/or the specification includes any measurable increase to achieve a desired result.

The term “effective,” as that term is used in the specification and/or claims, means adequate to accomplish a desired, expected, or intended result.

### WORKING EXAMPLES

The following working examples are provided to demonstrate preferred embodiments of the invention, but of course, should not be construed as in any way limiting the scope of the present invention. Further, it should be appreciated by those of skill in the art that the techniques and/or procedures disclosed in the examples represent techniques/procedures found by the inventor to function well in the practice of the invention, and thus can be considered to constitute preferred modes for its practice. However, those of skill in the art should, in light of the present disclosure, appreciate that many changes can be made to the specific embodiments which are disclosed and still obtain a like or similar result without departing from the spirit and scope of the invention. (Ingredients of various compositions/formulations in the disclosure herein are not listed in any predominant order unless otherwise indicated)

### Example 1

An example of the present invention was made by first producing eight mixtures called phase A, phase B, phase C, phase D, phase E, phase F, phase G and phase H. Phase A was comprised of the following components. INCI stands for International Nomenclature of Cosmetic Ingredients.

TABLE 1.1.
Composition of Phase A

| Ingredient | | | % by |
|---|---|---|---|
| (Trade Name) | INCI Designation | Supplier | weight |
| Water | Water (Aqua) | Local | 46.511 |
| Zemea Propanediol | Propanediol | Dupont/Essential | 2.000 |
| | | Ing. | |
| Disodium EDTA | Disodium EDTA | Dow | 0.100 |
| | | Chemical/Univar | |
| Sepimax Zen | Polyacrylate | Seppic | 1.500 |
| | Crosspolymer-6 | | |

TABLE 3
Composition of Phase B

| Ingredient | | | % by |
|---|---|---|---|
| (Trade name) | INCI Designation | Supplier | weight |
| Sepiplus S | Hydroxyethylacrylate/ | Seppic | 3.500 |
| | Sodium | | |
| | Acryloyldimethyl | | |
| | Taurate Copolymer | | |
| | Polyisobutene PEG-7 | | |
| | Trimethylolpropane | | |
| | Coconut Ether | | |
| Gransil GCM-5 | Cyclopentasiloxane | Grant | 4.000 |
| | Polysilicone-11 | | |
| Vitamin E Acetate | Tocopheryl Acetate | JEEN | 0.100 |
| — | | | |
| Xiameter(R) PMX- | Cyclopentasiloxane | Dow Corning/ | 3.000 |
| 0245 | | Univar | |
| Arlacel 165-PW- | Glyceryl Stearate | Croda | 1.500 |
| (AP) | PEG-100 Stearate | | |
| Jojoba Esters-15 | Jojoba Esters | Desert Whale | 5.000 |
| Refined Shea | Butyrospermum | Brenntag | 1.000 |
| Butter PC | Parkii (Shea) Butter | | |
| Lipowax D | Cetearyl Alcohol | Vantage | 2.000 |
| | Ceteareth-20 | | |
| Neossance | Squalane | Centerchem/ | 3.000 |
| Squalane | | Amyris | |

TABLE 4
Composition of Phase C

| Ingredient | | | % by |
|---|---|---|---|
| (Trade Name) | INCI Designation | Supplier | Weight |
| Water | Water (Aqua) | Local | 7.500 |
| Sodium Chloride | Sodium Chloride | Morton Salt | 0.250 |
| Fine, USP | | | |
| Potassium Chloride | Potassium Chloride | UPI | 0.040 |
| USP | | | |
| Magnesium | Magnesium | UPI | 0.010 |
| Chloride USP | Chloride | | |
| Calcium Chloride | Calcium Chloride | UPI | 0.014 |
| USP | | | |
| Magnesium Sulfate | Magnesium Sulfate | UPI | 0.010 |
| USP | | | |
| Potassium | Potassium | UPI | 0.006 |
| Phosphate | Phosphate | | |
| Monobasic USP | Monobasic | | |
| Sodium | Sodium | UPI | 0.036 |
| Bicarbonate USP | Bicarbonate | | |
| Sodium Phosphate | Sodium Phosphate | UPI | 0.003 |
| Dibasic Anhydrous | Dibasic Anhydrous | | |
| USP | | | |

TABLE 5
Composition of Phase D

| Ingredient | | | |
|---|---|---|---|
| (Trade Name | INCI Designation | Supplier | % by Weight |
| Primalhyal 3K | Hydrolyzed | Soliance | 0.500 |
| | Hyaluronic Acid | | |
| Water | Water (Aqua) | Local | 5.000 |

TABLE 6
Composition of Phase E

| Ingredient | | | % by |
|---|---|---|---|
| (Trade Name) | INCI Designation | Supplier | Weight |
| Skinmimics | Ceteareth-25 | Evonik/Univar | 0.750 |
| | Gycerin, Cetyl | | |
| | Alcohol, Behenic | | |
| | Acid, Ceramide NP, | | |
| | Ceramide NS, | | |
| | Ceramide EOS, | | |
| | Ceramide EOP, | | |
| | Ceramide AP, | | |
| | Caprooyl | | |
| | Phytosphingosine, | | |
| | Carprooyl | | |
| | Sphingosine | | |
| NAB Willowbark | Water Salix Nigra | Lonza/Dewolf | 5.000 |
| Extract | (Willow) Bark | | |
| | Extract | | |
| Sohakuhi BG | Butylene Glycol | Charkit | 2.000 |
| | Morus Alba Root | | |
| | Extract | | |
| Dextrose Anydrous | Glucose | Spectrum | 2.000 |
| Granular USP | | | |
| L-Arginine | Arginine | Kyowa USA | 0.300 |
| L-Glutathione | L-Glutathione | Kyowa | 0.620 |
| Reduced | | | |
| Scavenox GTE | Water, Glycerin, | Biocogent | 1.000 |
| | Camellia Sinensis | | |
| | Leaf Extract | | |

Phase F was comprised of Euxyl PE, (INCI designation Phenoxyethanol Ethylhexylglycerin) (Supplier Schulke) % by weight 1.000.
Phase G was comprised of Ascorbyl Glucoside supplied by Hayashibara/DKSH, % by weight 0.250.
Phase H was 20% solution of citric acid in water used as a buffering agent % by weight 0.500.
Manufacturing of this composition was as follows:
Step 1—The Sepimax Zen was dispersed in water and heated to between 65 and 70° C. and the remaining components of phase A were added while heating and mixing.
Step 2—The components of Phase B were mixed together and warmed to between 65 and 70° C. The composition of Phase B was mixed together with the composition of Phase A and homogenized for 5-10 minutes at 4000 RPM or until the mixture became uniform. The mixture was then cooled to about 44-50° C. with mixing.
Step 3—The salts of Phase C were dissolved in water and the resultant solution was added to the mixture produced by Step 2.
Step 4—Primalhyal 3K of Phase D was dissolved in water and added to the composition of Step 3.
Step 5—Each of the components of Phase E were added one at a time to the composition of Step 4. Each component was thoroughly dissolved in the mixture before the next one was added.
Step 6—Euxyl PE 9010 was then added to the composition of Step 5 and mixed until it was uniformly dispersed in the composition.
Step 7—Ascorbyl-2-Glucoside was then added to the composition of Step 6 and thoroughly mixed.
Step 8—The 20% solution of citric acid was added to the composition of Step 7 until a pH of 3.8 to 4.2 was obtained.

The facial cream so prepared from the above process has film forming and light reflecting qualities.

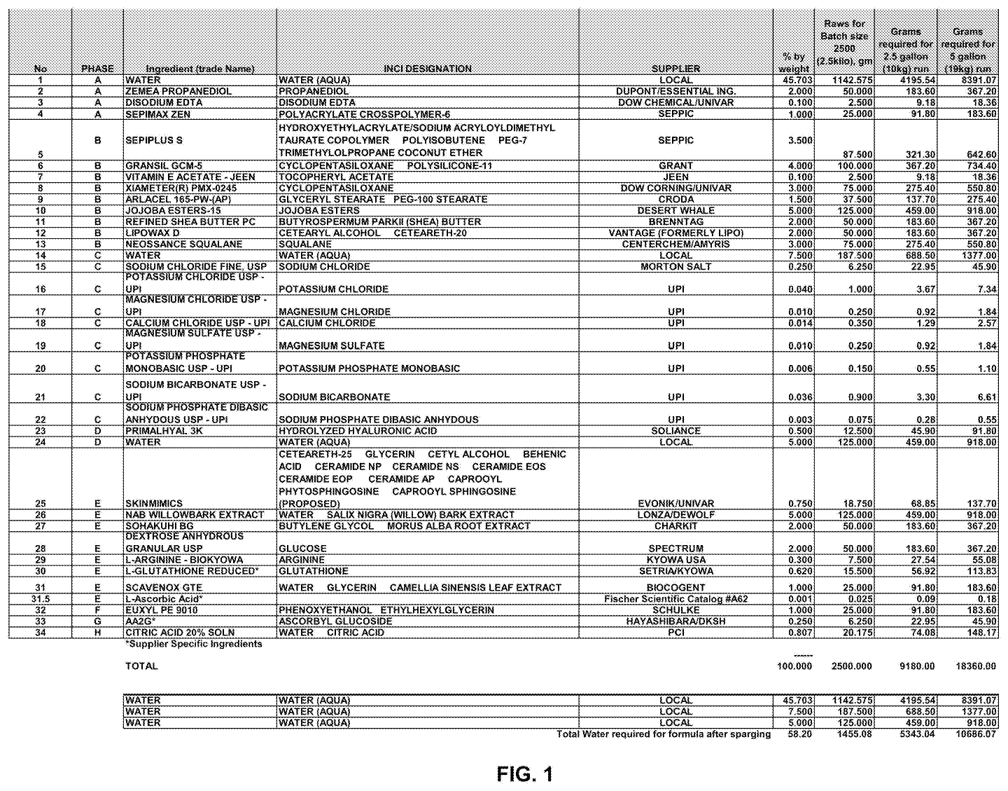

### Example 2: FIG. 1 Shows an Example of Dermatological Cream Composition

MANUFACTURING of this composition was as follows:

Prior to beginning—All the water needed for the entire formula needs to be sparged per common laboratory instructions.
PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

### Specs:

### Viscosity Range: 110,000 CPS-99,000 CPS

### pH Range: 3.8-4.2

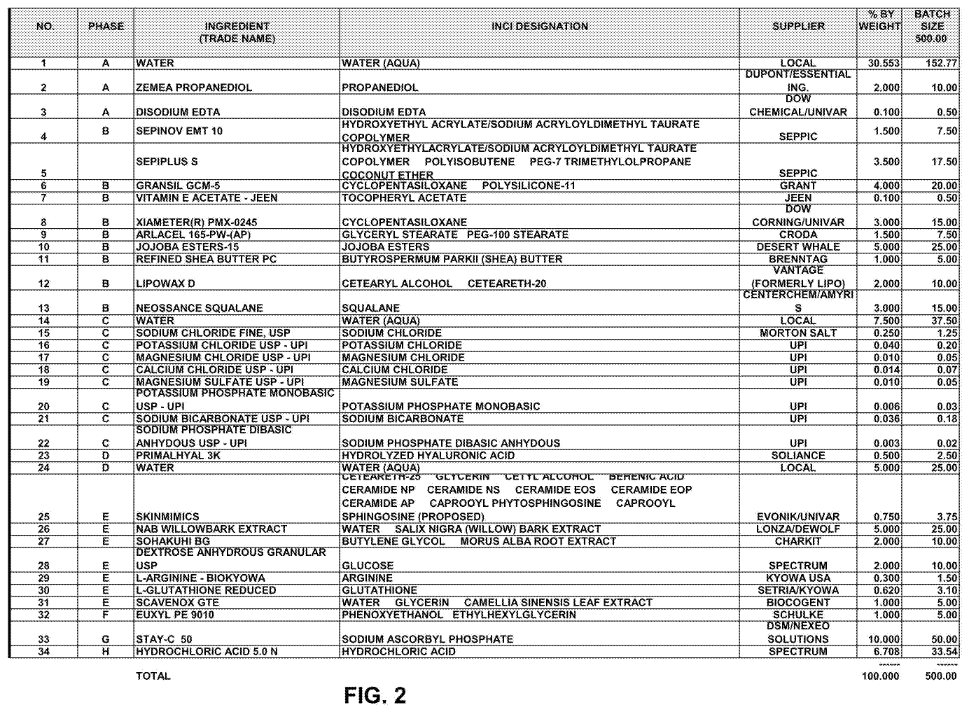

### Example 3: FIG. 2 Shows Another Example of Dermatological Cream Composition

MANUFACTURING of this composition was as follows:

PHASE A: Mix PHASE A together until uniform and heat to 65-70 C.
PHASE B: Mix PHASE B together until uniform warm to 65-70 C. Add PHASE B to PHASE A and mix until uniform. Homogenize for 5 mins at 4000 rpm for 5 mins or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Slowly add PHASE G to batch mix until uniform.
PHASE H Adjust pH to 3.8-4.5 with PHASE H.

### Other Information: T-C @5 rpm for 1 Minute

### pH: 4.37 Specific Gravity: Viscosity (CPS): 56,000

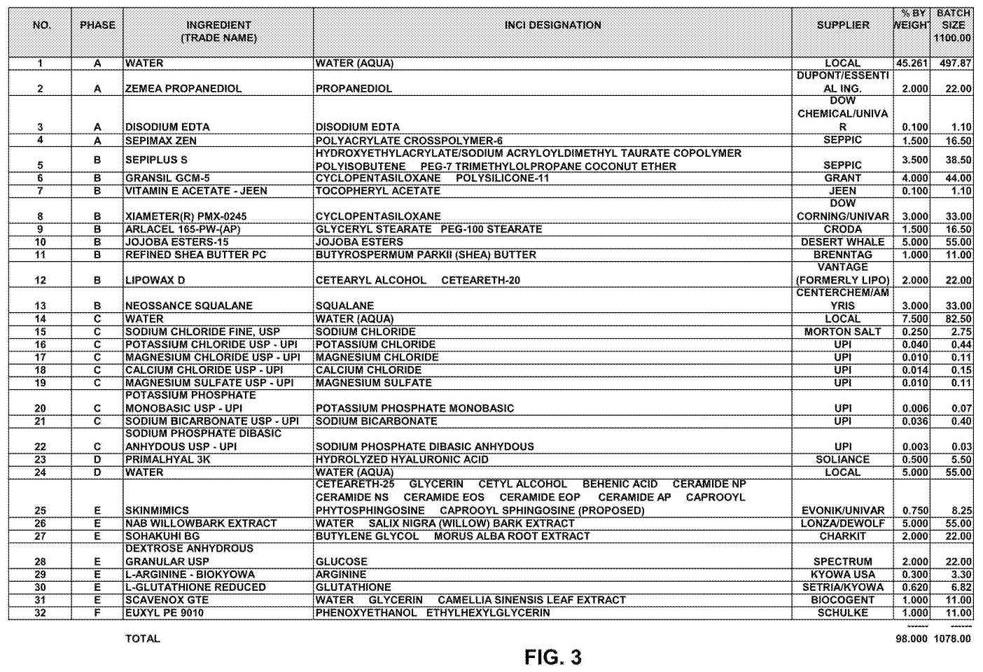

### Example 4: FIG. 3 Shows Another Example of Dermatological Cream Composition

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform. Cool with mixing to room temperature. Other information: t-c @5 rpm for 1 minute
Ph: 5.02 specific gravity: viscosity (cps): 110,000

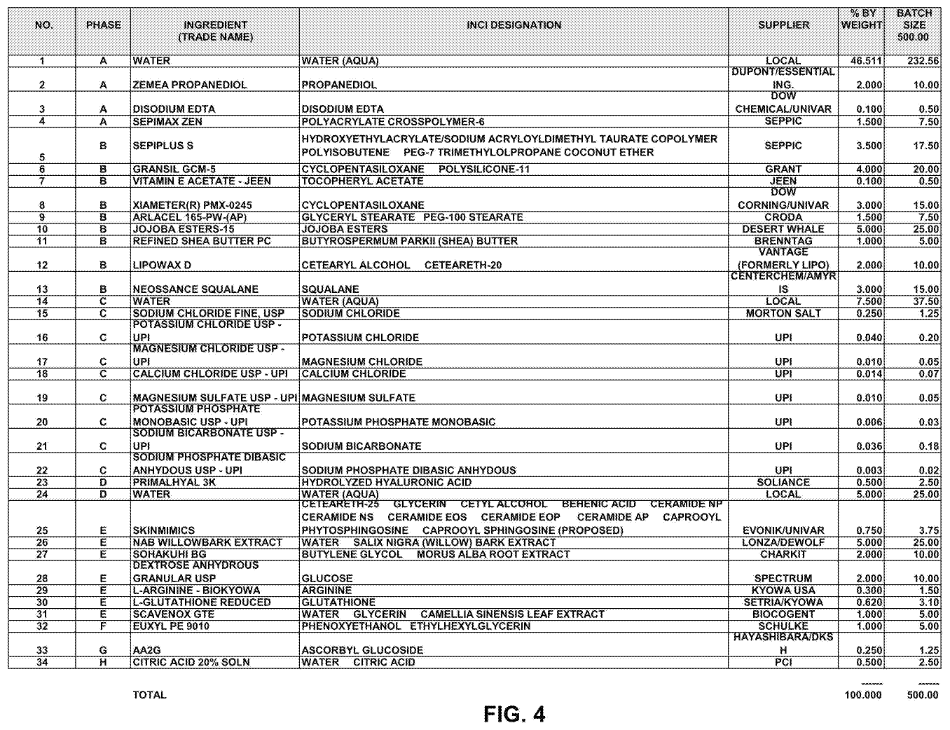

### Example 5: FIG. 4 Shows Another Example of Dermatological Cream Composition (with 0.25% AA2G)

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45° C.-50° C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.23; Specific gravity: viscosity (CPS): 110,000 CPS

OTHER INFORMATION: T-C @5 RPM for 1 minute.

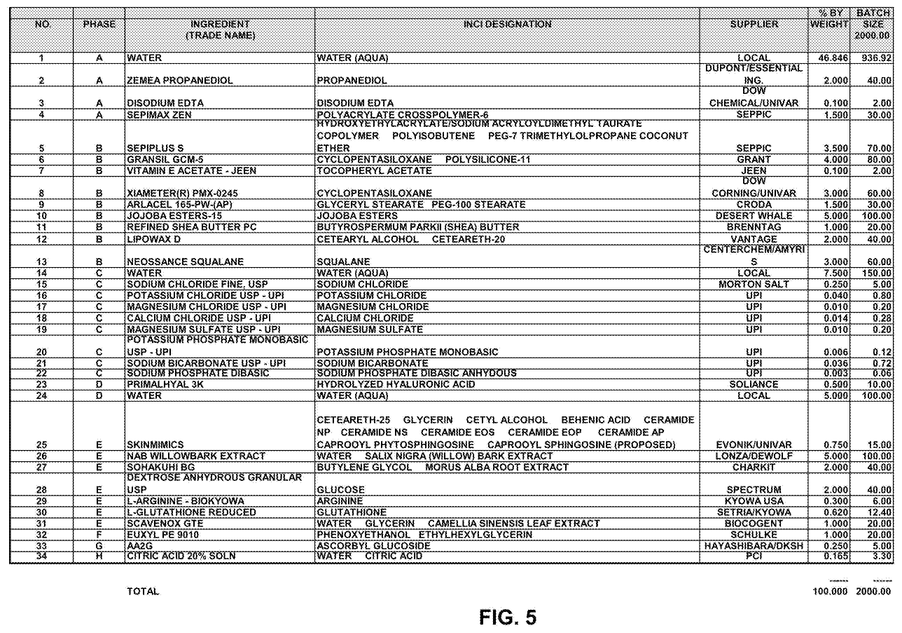

### Example 6: FIG. 5 Shows Another Example of Dermatological Cream Composition

MANUFACTURING of this composition was as follows:

PHASE A: Disperse the SEPIMAX ZEN in water and heat to 65-70° C. Add remainder of PHASE A while heating and mixing.
PHASE B: Mix PHASE B together until uniform warm to 65-70° C. Add PHASE B to PHASE A and mix until uniform. Homogenize for 5-10 mins at 4000 rpm or until uniform. Switch to mixer and cool with mixing to 45° C.-50° C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.18; Specific gravity: viscosity (CPS): 117,000 CPS

OTHER INFORMATION: T-C @5 RPM for 1 minute.

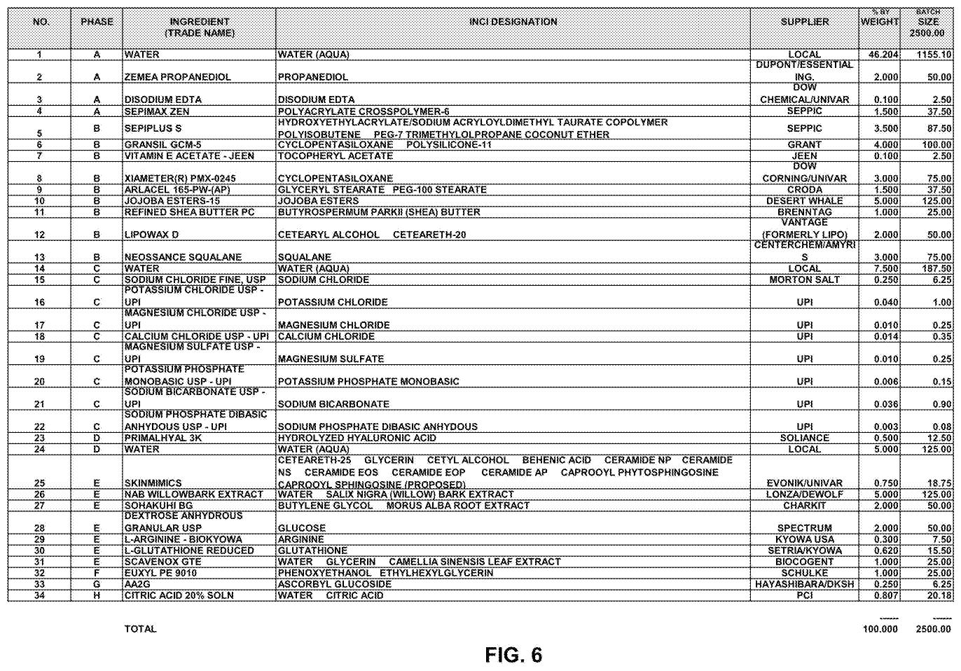

### Example 7: FIG. 6 Shows Another Example of Dermatological Cream Composition

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45° C.-50° C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.29 Specific gravity: viscosity (CPS): 102,000 CPS

OTHER INFORMATION: T-C @5 RPM for 1 minute.

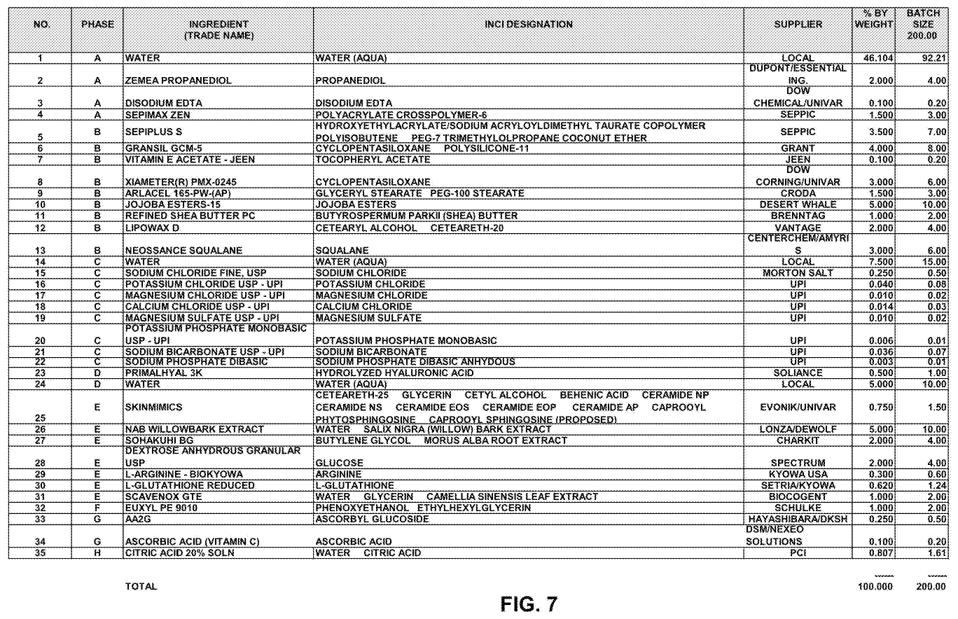

### Example 8: FIG. 7 Shows Another Example of Dermatological Cream Composition (with 0.1% Ascorbic Acid)

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.38 Specific gravity: viscosity (CPS): 134,000 CPS

OTHER INFORMATION: T-C @5 RPM for 1 minute.

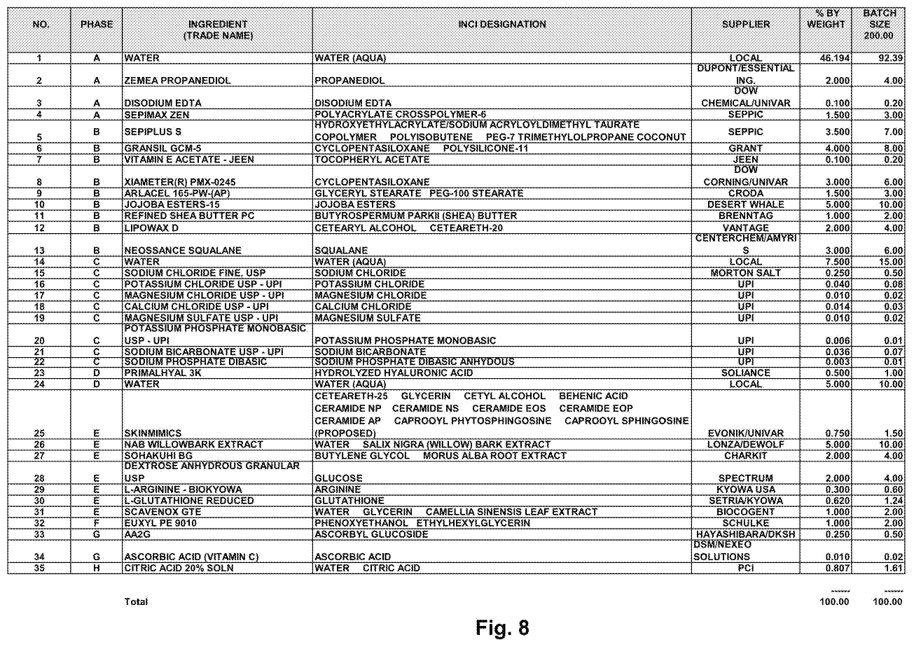

### Example 9: FIG. 8 Shows Another Example of Dermatological Cream Composition (with 0.01% Ascorbic Acid)

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.58 Specific gravity: viscosity (CPS): 162,000 CPS

OTHER INFORMATION: T-C @5 RPM for 1 minute.

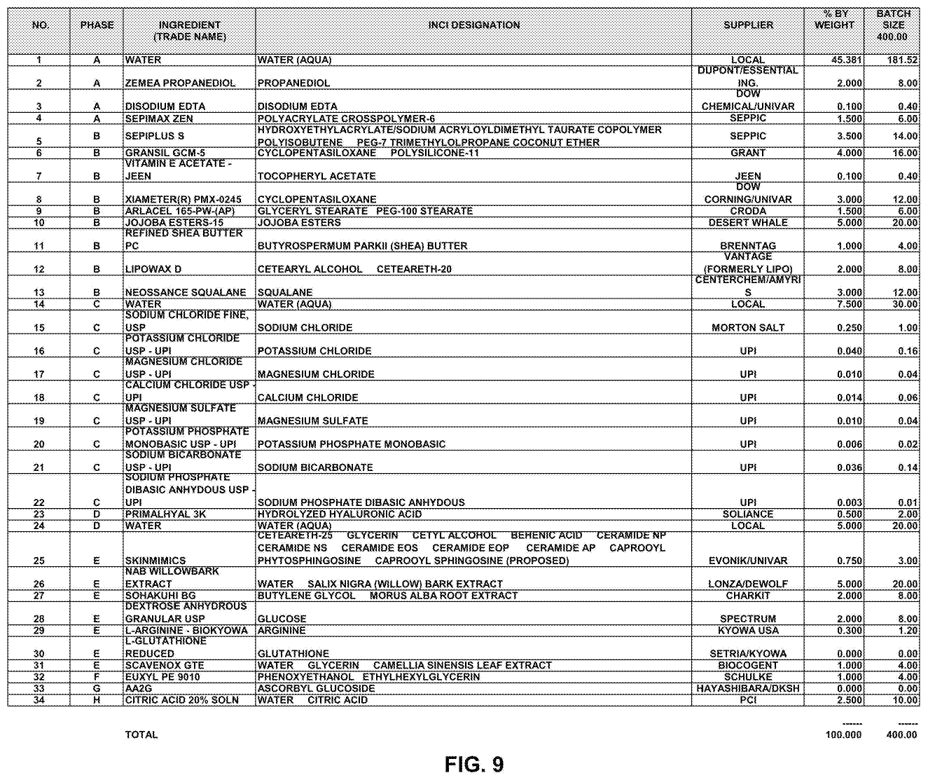

### Example 10: FIG. 9 Shows Another Example of Dermatological Cream Composition (without AA2G and Glutathione)

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient. Note: Glutathione not added to this Example.
PHASE F: Add PHASE F to batch and mix until uniform.
PHASE G: In this Example no Phase G is added.
PHASE H: Adjust pH to 3.8-4.2 with PHASE H.

pH: 4.30; Specific gravity: viscosity (CPS): 99.000 CPS

OTHER INFORMATION: T-C @5 RPM for 1 minute.

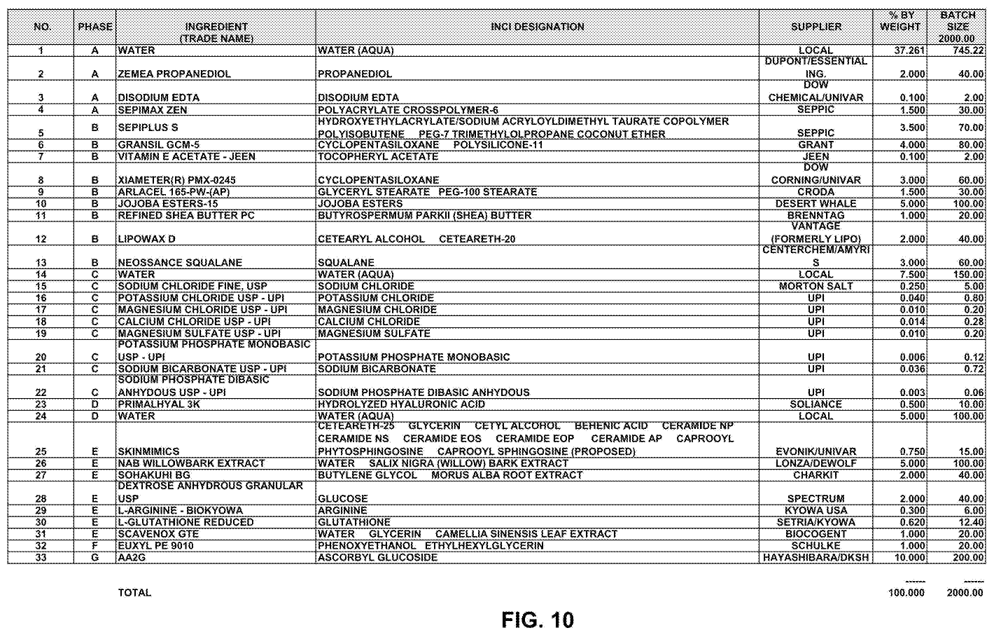

### Example 11: FIG. 10 Shows Another Example of Dermatological Cream Composition (with 10% AA2G)

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
Adjust pH TO 3.5-4.0 with 50% sodium hydroxide.

pH: 2.79; Specific gravity: viscosity (CPS): 115,000 CPS

OTHER INFORMATION: T-C @5 RPM for 1 minute.

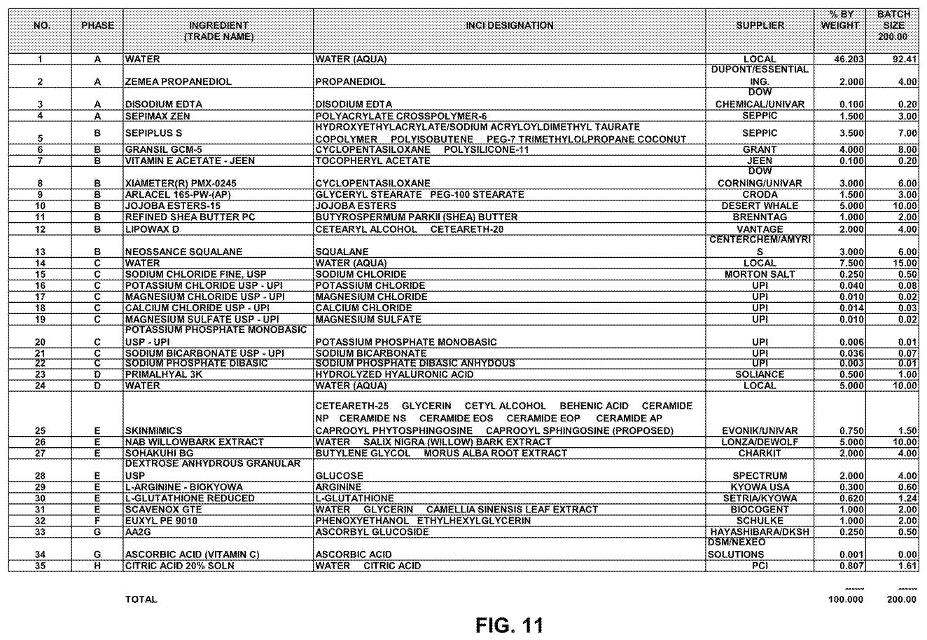

### Example 12: FIG. 11 Shows Another Example of Dermatological Cream Composition (with 0.001% Ascorbic Acid)

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.45 Specific gravity: viscosity (CPS): 127,000 CPS

OTHER INFORMATION: T-C @5 RPM for 1 minute.

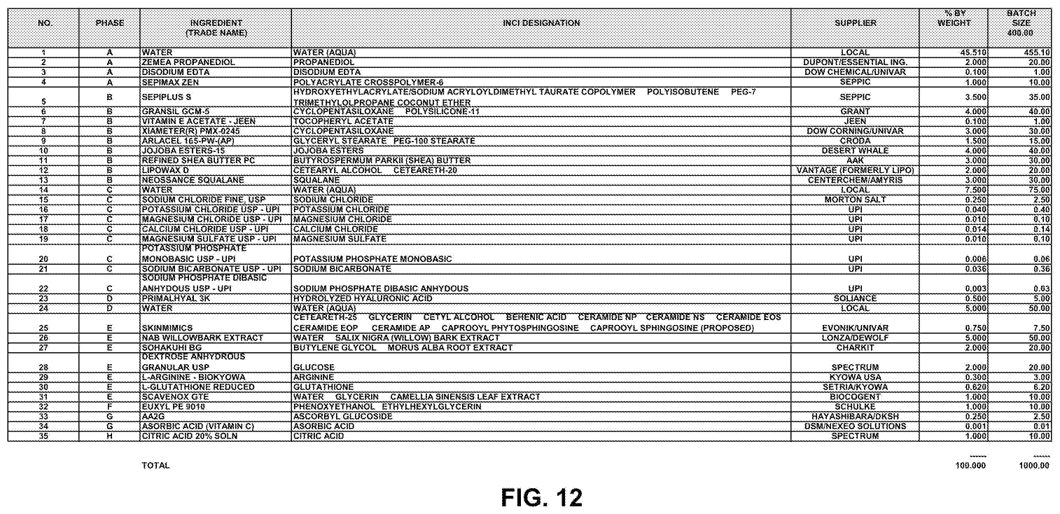

### Example 13: FIG. 12 Shows Another Example of Dermatological Cream Composition

MANUFACTURING of this composition was as follows:

PHASE A: Disperse the SEPIMAX ZEN in water and heat to 65-70 C. Add remainder of PHASE A while heating and mixing.
PHASE B: Mix PHASE B together until uniform warm to 65-70 C. Add PHASE B to PHASE A and mix until uniform. Homogenize for 5-10 mins at 4000 rpm or until uniform. Switch to mixer and cool with mixing to 45 C-50 C.
PHASE C: Dissolve all the salts in PHASE C in the water and add to batch.
PHASE D: Dissolve PRIMALHYAL in water and add to batch.
PHASE E: Add PHASE E one ingredient at a time to batch and make sure each one dissolves before adding the next ingredient.
PHASE F: Add PHASE F to batch and mix until uniform.
PHASE G: Add PHASE G to batch and mix until uniform.
PHASE H: Adjust pH to 3.8-4.2 with PHASE H.

pH: 4.29 Specific gravity: viscosity (CPS): 106,000 CPS

OTHER INFORMATION: T-C @5 RPM for 1 minute.

### Example 14: Antimicrobial Preservative Efficacy Test

Procedure Summary:

The CTFA Antimicrobial Effectiveness Test consisted of challenging test samples with mixed inoculum pools of the test organisms indicated below. The bacterial inoculum pool consisted of Staphylococcus aureus, ATCC 6538, Escherichia coli, ATCC 8739, Pseudomonas aeruginosa, ATCC 9027. The yeast mold inoculum pool consisted of Candida albicans, ATCC 10231 and Aspergillus brasiliensis, ATCC 16404. The changes in microbial population were determined at specified time intervals of 2, 7, 14, 21 and 28 days for each microorganism pool (bacterial or yeast/mold) to recover any surviving test organisms.

Test Organisms:
Staphylococcus aureus, ATCC 6538
Escherichia coli, ATCC 8739
Pseudomonas aeruginosa, ATCC 9027
Candida albicans, ATCC 10231
Aspergillus brasiliensis, ATCC 16404 (formerly Aspergillus niger)

### Results:

Table 7 and Table 8 shows the results for the Antimicrobial Effectiveness Test and Neutralizer Effectiveness Test.

### Conclusion:

This study demonstrated that the preservative system for the test sample meets the acceptance criteria per the CTFA Antimicrobial Preservative Effectiveness Method.

Results:

TABLE 7
Antimicrobial Effectiveness Test Results

| | Staphylococcus | Escherichia | Pseudomonas | Candida | Aspergillus |
|---|---|---|---|---|---|
| | aureus | coli | aeruginosa | albicans | brasiliensis |
| | ATCC 6538 | ATCC 8739 | ATCC 9027 | ATCC 10231 | ATCC 16404 |
| | CFU/g | CFU/g | CFU/g | CFU/g | CFU/g |
| Initial | 1.3 × 10⁶ | 1.7 × 10⁶ | 1.5 × 10⁶ | 1.0 × 10⁶ | 7.1 × 10⁵ |
| Inoculum | | | | | |
| Level | | | | | |

| | Total Bacteria/Inoculum | Total Yeast/Mold Inoculum |
|---|---|---|
| | Pool | Pool |
| | CFU/g | CFU/g |
| | (% Reduction) | (% Reduction) |
| Initial Pooled | 1.6 × 10⁶ | 7.71 × 10⁵ |
| Inoculum Level | | |
| Day 2 Count: | <10 | <10 |
| | >99 9 | >99.9 |
| Day 7 Count: | <10 | <10 |
| | >99.9 | >99.9 |
| Day 14 Count: | <10 | <10 |
| | >99.9 | >99.9 |
| Day 21 Count: | <10 | <10 |
| | >99.9 | >99.9 |
| Day 28 Count: | <10 | <10 |
| | >99.9 | >99.9 |

$$\begin{matrix}Calculation \text{:} \% Reduction = \underline{\begin{matrix}( Initial count ( CFU / g ) - Count at \\ sampling time ( CFU / g ) \times 100\end{matrix} } \\ Initial Count ( CFU / g )\end{matrix}$$

Acceptance Criteria for CTFA Antimicrobial Preservative Effectiveness:
Bacteria: There should be at least a 99.9% reduction of vegetative bacteria within 7 days following each challenge and no increase for the duration of the test period.
Yeast and Molds: There should be at least a 90% reduction of yeasts and molds within 7 days following each challenge and no increase for the duration of the test period.
RESULTS: PASS

TABLE 8
Neutralizer Effectiveness Test Results

| | Neutralizer Efficacy |
|---|---|
| | Log Difference at 1:10 |
| Test Organisms | dilution |
| Total Bacterial Inoculum Pool | 0.11 |
| Total Yeast Mold Inoculum Pool | 0.13 |

Dilution at which Neutralization was achieved: 1:10
Neutralizing Diluent: O/E Neutralizing Broth
Acceptance Criteria for Neutralizer Effectiveness:
The plate count for the neutralizer/test sample broth and the peptone buffer “control” (viability control) must be within 0.5 log of each other in order for the neutralizer to be validated for use with the preserved product.
RESULT: PASS

### Example 15: Dermatological Cream Composition Stability Testing

| | | | | G.R. | TARE | NET | % WT |
|---|---|---|---|---|---|---|---|
| INITIAL | pH | VISCOSITY | APPEARANCE | WT. | WT. | WT. | Loss |
| 1 | | | | 24.68 | 13.40 | 11.28 | |
| 2 | | | | 23.51 | 13.40 | 10.11 | |
| 3 | | | | 24.23 | 13.40 | 10.83 | |
| 4 | | | | 25.00 | 13.40 | 11.60 | |
| 5 | | | | 24.76 | 13.40 | 11.36 | |
| 6 | | | | 24.70 | 13.40 | 11.30 | |
| 7 | | | | 25.16 | 13.40 | 11.76 | |
| 8 | | | | 23.28 | 13.40 | 9.88 | |
| 9 | | | | 24.94 | 13.40 | 11.54 | |
| 10 | | | | 24.31 | 13.40 | 10.91 | |
| 11 | | | | 24.57 | 13.40 | 11.17 | |
| 12 | | | | 24.80 | 13.40 | 11.40 | |
| 13 | | | | 24.80 | 13.40 | 11.40 | |
| 14 | | | | 23.44 | 13.40 | 10.04 | |
| 15 | | | | 24.98 | 13.40 | 11.58 | |
| 16 | | | | 24.77 | 13.40 | 11.37 | |
| 17 (Glass Jar) | | | | | | | |
| 18 (Glass Jar) | | | | | | | |
| 19 (Glass Jar) | | | | | | | |
| 20 (Glass Jar) | | | | | | | |
| FREEZE/ | | | | | | | |
| THAW: | | | | | | | |
| CYCLE 1 | | | GOOD | | | | |
| CYCLE 2 | | | GOOD | | | | |
| CYCLE 3 | | | GOOD | | | | |
| 2 WEEKS @ RT: | | | | | | | |
| 1 | | | | 24.65 | 13.40 | 11.25 | 0.27% |
| 2 | | | | 23.50 | 13.40 | 10.10 | 0.10% |
| 3 | | | | 24.22 | 13.40 | 10.82 | 0.09% |
| 4 | | | GOOD | | | | |
| 5 | | | | 24.75 | 13.40 | 11.35 | 0.09% |
| 17 (Glass Jar) | 4.60 | 128,000 CPS | | | | | |
| 2 WKS @ 40 C.: | | | | | | | |
| 6 | | | | 24.65 | 13.40 | 11.25 | 0.44% |
| 8 | | | | 23.25 | 13.40 | 9.85 | 0.30% |
| 9 | | | | 24.90 | 13.40 | 11.50 | 0.35% |
| 10 | | | | 24.30 | 13.40 | 10.90 | 0.09% |
| 18 (Glass Jar) | 4.57 | 150,000 | CPS | GOOD | | | |
| 2 WKS @ 4 C.: | | | | | | | |
| 11 | | | | 24.55 | 13.40 | 11.15 | 0.18%* |
| 12 | | | | 24.79 | 13.40 | 11.39 | 0.09%* |
| 13 | | | | 24.77 | 13.40 | 11.37 | 0.26%* |
| 14 | | | | 23.44 | 13.40 | 10.04 | 0.00% |
| 15 | | | | GOOD | | | |
| 19(GlassJar) | 4.59 | 128,800 | CPS | GOOD | | | |
| COMMENTS: PACKAGE - SATISFACTORY *Slightly high % weight loss at 4C probably due to | | | | | | | |
| overfilling, continue to monitor | | | | | | | |
| PRODUCT - SATISFACTORY | | | | | | | |
| 4 WEEKS @ RT.: | | | | | | | |
| 1 | | | | 24.65 | 13.40 | 11.25 | 0.27% |
| 2 | | | | 23.50 | 13.40 | 10.10 | 0.10% |
| 3 | | | | 24.22 | 13.40 | 10.82 | 0.09% |
| 4 | | | GOOD | | | | |
| 5 | | | | 24.75 | 13.40 | 11.35 | 0.09% |
| 17(GlassJar) | 4.63 | 120,000 CPS | GOOD | | | | |
| 4 WEEKS @ 40C.: | @ | | | | | | |
| 6 | | | | 24.63 | 13.40 | 11.23 | 0.62% |
| 7 | | | GOOD | | | | |
| 8 | | | | 23.25 | 13.40 | 9.85 | 0.30% |
| 9 | | | | 24.87 | 13.40 | 11.47 | 0.61% |
| 10 | | | | 24.28 | 13.40 | 10.88 | 0.27% |
| 18 (Glass Jar) | 4.63 | 160,000 CPS | GOOD | | | | |
| 4WEEKS @ 4 C.: | | | | | | | |
| 11 | | | | 24.55 | 13.40 | 11.15 | 0.18%* |
| 12 | | | | 24.78 | 13.40 | 11.38 | 0.18%* |
| 13 | | | | 24.76 | 13.40 | 11.36 | 0.35%* |
| 14 | | | | 23.44 | 13.40 | 10.04 | 0.00% |
| 15 | | | GOOD | | | | |
| 19 (Glass Jar) | 4.63 | 124,000 CPS | GOOD | | | | |
| COMMENTS PACKAGE- SATISFACTORY *Slightly high % weight loss at 4C probably due to | | | | | | | |
| overfilling, continue to monitor | | | | | | | |
| PRODUCT - SATISFACTORY | | | | | | | |
| 8 WEEKS @ RT: | | | | | | | |
| 1 | | | | 24.64 | 13.40 | 11.24 | 0.35% |
| 2 | | | | 23.50 | 13.40 | 10.10 | 0.10% |
| 3 | | | | 24.22 | 13.40 | 10.82 | 0.09% |
| 4 | | | GOOD | | | | |
| 5 | | | | 24.74 | 13.40 | 11.34 | 0.18% |
| 17 (Glass Jar) | 4.62 | 122,000 CPS | GOOD | | | | |
| 8 WEEKS @ 40 C.: | | | | | | | |
| 6 | | | | 24.60 | 13.40 | 11.20 | 0.88% |
| 7 | | | GOOD** | | | | |
| 8 | | | | 23.23 | 13.40 | 9.83 | 0.51% |
| 9 | | | | 24.83 | 13.40 | 11.43 | 0.95% |
| 10 | | | | 24.26 | 13.40 | 10.86 | 0.46% |
| 18 (Glass Jar) | 4.61 | 150,000 CPS | GOOD** | | | | |
| 8 WEEKS @ 4 C.: | | | | | | | |
| 11 | | | | 24.54 | 13.40 | 11.14 | 0.27%* |
| 12 | | | | 24.77 | 13.40 | 11.37 | 0.26%* |
| 13 | | | | 24.75 | 13.40 | 11.35 | 0.44%* |
| 14 | | | | 23.44 | 13.40 | 10.04 | 0.00% |
| 15 | | | GOOD | | | | |
| 19 (Glass Jar) | 4.61 | 120,000 CPS | GOOD | | | | |
| COMMENTS: PACKAGE- SATISFACTORY *Slightly high % weight loss at 4C probably due to | | | | | | | |
| overfilling, continue to monitor | | | | | | | |
| PRODUCT-SATISFACTORY **Slightly yellow with a slightly stronger characteristic odor. | | | | | | | |
| 12 WEEKS @ RT: | | | | | | | |
| 1 | | | | 24.64 | 13.40 | 11.24 | 0.35% |
| 2 | | | | 23.49 | 13.40 | 10.09 | 0.20% |
| 3 | | | | 24.21 | 13.40 | 10.81 | 0.18% |
| 4 | | | GOOD | | | | |
| 5 | | | | 24.73 | 13.40 | 11.33 | 0.26% |
| 17(GlassJar) | 4.61 | 122,000 CPS | GOOD | | | | |
| 12 WEEKS @ 40 C.: | | | | | | | |
| 6 | | | | 24.56 | 13.40 | 11.16 | 1.24% |
| 7 | | | GOOD** | | | | |
| 8 | | | | 23.19 | 13.40 | 9.79 | 0.91% |
| 9 | | | | 24.79 | 13.40 | 11.39 | 1.30% |
| 10 | | | | 24.22 | 13.40 | 10.82 | 0.82% |
| 18(GlassJar) | 4.58 | 150,000 CPS | GOOD** | | | | |
| 12 WEEKS @ 4 C.: | | | | | | | |
| 11 | | | | 24.53 | 13.40 | 11.13 | 0.36%* |
| 12 | | | | 24.76 | 13.40 | 11.36 | 0.35%* |
| 13 | | | | 24.74 | 13.40 | 11.34 | 0.53%* |
| 14 | | | | 23.44 | 13.40 | 10.04 | 0.00% |
| 15 | | | GOOD | | | | |
| 19(GlassJar) | 4.59 | 124,000 CPS | GOOD | | | | |

FINAL COMMENTS: PACKAGE - SATISFACTORY *Very slightly high % weight loss at 4C probably
due to initial overfilling.
PRODUCT - SATISFACTORY **Slight tan color with a slightly stronger characteristic odor.

### Example 16: Stability Testing of Various Dermatological Cream Compositions of the Invention

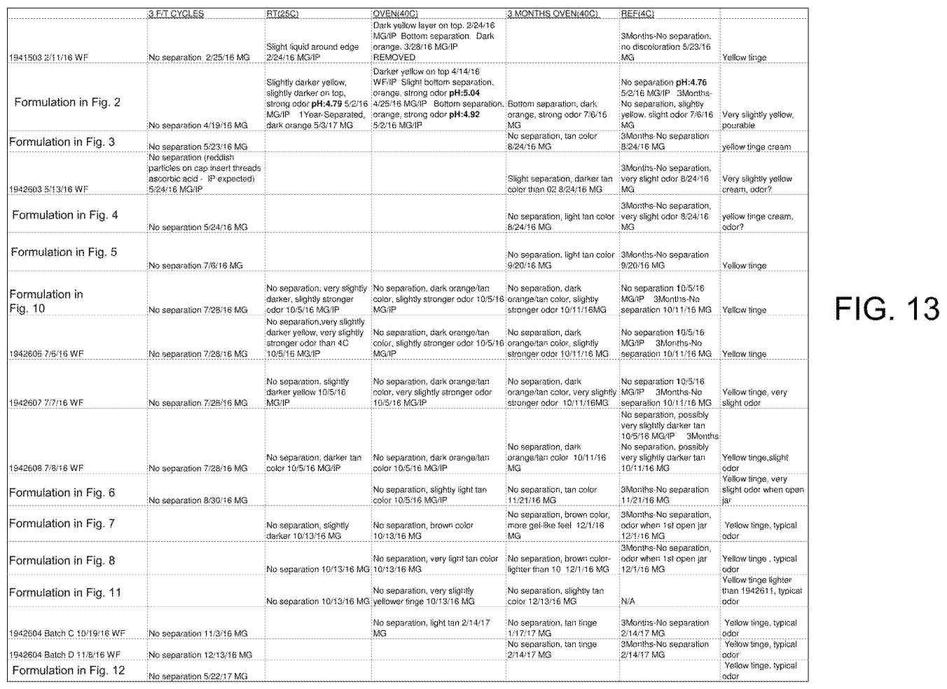

Compositions were exposed to various conditions and evaluated for the tolerance of those conditions. The conditions included three cycles of freeze and thaw, storage at room temperature, storage in a 40 C oven, storage in refrigeration at 4 C and storage in a 40 C oven for three months. FIG. 13 tabulates the results of the study.

### Example 17: Clinical Study to Demonstrate the Effect of a Facial Cream Treatment on Skin Health

Clinical Evaluation: An extensive “statistically powered” clinical evaluation was made to demonstrate the effect of cream composition on facial skin health and appearance. Assessments were made using a combination of non-invasive technological-based skin measurements to evaluate skin elasticity, function, color and texture. In addition to dermatologist evaluation, study participant self-evaluation and photographic documentation were also performed at intervals throughout the 12 week study.

Study participants were female subjects 35-65 years of age with mild to moderate photoaging. The following parameters were evaluated and reported throughout the course of the study: Skin elasticity, skin firmness, fine lines, wrinkles, skin tone, laxity, skin color, tactile smoothness, textural smoothness, pigmentation, radiance, luminosity, skin hydration, and overall appearance. Statistical Methods: A two-tailed Mann Whitney t-test was used to analyze the nonparametric data sets (investigator efficacy and tolerability, subject efficacy and tolerability) with significance set at p<0.05. The noninvasive parametric numerical data (TEWL, corneometry, elasticity, colorimetry) was analyzed with a Student t test with significance set at p<0.05. A longitudinal analysis was performed for the monadic study with each timepoint compared to baseline.

Summary of Results: The cream composition of Example 2 delivered astounding results over the course of the 12 week study. 100% of women reported improvements in overall skin appearance. There was a highly statistically significant improvement in skin health and statistically significant measureable increases in skin elasticity and skin luminosity and statistically significant measureable decreases in ruddy, yellow and brown tones. Within 8 weeks there was a statistically significant reported improvement in fine lines and wrinkles, skin firmness, textural smoothness and radiance. By 12 weeks, all measured parameters were highly statistically significant indicating that the cream is highly effective as an anti-aging skin treatment.

### Example 18: Dermatological Toner Composition without Preservative(s)

TABLE 18.1
Toner formula with no preservative, at a pH of ~6.0,
Density of Toner = 1.002 g/ml.

| | | Quantity (% | Batch quantity | mM |
|---|---|---|---|---|
| S. No. | Ingredient | w/w) | (1002 g)(1L) | Concentration |
| 1 | Purified water | 99.117 | 93.16 | 55128.757 |
| 2 | Sodium chloride | 0.800 | 8.016 | 137.159 |
| 3 | Potassium | 0.040 | 0.401 | 5.379 |
| | chloride | | | |
| 4 | Magnesium | 0.010 | 0.100 | 0.492 |
| | Chloride 6H₂O | | | |
| 5 | Calcium | 0.014 | 0.140 | 0.952 |
| | chloride. 2H₂O | | | |
| 6 | Magnesium | 0.010 | 0.100 | 0.406 |
| | sulfate. 7H₂O | | | |
| 7 | Potassium | 0.006 | 0.060 | 0.441 |
| | phosphate | | | |
| | monobasic | | | |
| 8 | Sodium | 0.0027 | 0.027 | 0.190 |
| | phosphate | | | |
| | dibasic | | | |
| | anhydrous | | | |
| | Total: | 100.00 | 1002.00 | |

The above Toner formulation was manufactured as follows:
Ingredient 1 was added into a beaker.
Ingredients 2-8 were very slowly added into the beaker, while mixing vigorously at 250-1000 rpm using 3″ propeller blade until homogeneous.
pH was measured. The pH was adjusted to 6.0±0.25, using either 5N HCl or 5N NaOH as needed.

### Example 19: Dermatological Toner Composition with Preservative(s)

TABLE 19.1
Toner formula with preservative, at a pH of ~6.0, Density of Toner =
1.002g/mL.

| | | | Batch | | |
|---|---|---|---|---|---|
| | | | quantity | | |
| | | Quantity | (1002 g) | | |
| S. No | Ingredient | (% w/w) | (1L) | mM Concentration | |
| 1 | Purified water | 98.117 | 983.16 | 54573.673 | |
| 2 | Sodium chloride | 0.800 | 8.016 | 137.159 | |
| 3 | Potassium | 0.040 | 0.401 | 5.379 | |
| | chloride | | | | |
| 4 | Magnesium | 0.010 | 0.100 | 0.492 | |
| | chloride. 6H₂O | | | | |
| 5 | Calcium chloride | 0.014 | 0.140 | 0.952 | |
| | 2H₂O | | | | |
| 6 | Magnesium | 0.010 | 0.100 | 0.406 | |
| | sulfate. 7H₂O | | | | |
| 7 | Potassium | 0.006 | 0.060 | 0.441 | |
| | phosphate | | | | |
| | monobasic | | | | |
| | Sodium phosphate | 0.003 | 0.027 | 0.190 | |
| | dibasic anhydrous | | | | |
| 9 | Preservative(s): | 1.000 | 10.020 | Caprylyl | 113.705 |
| | Caprylyl Glycol, | | | Glycol | |
| | Phenoxyethanol | | | Phenoxy | 29.009 |
| | and Propylene | | | Ethanol | |
| | Glycol | | | Propylene | 52.671 |
| | | | | Glycol | |
| | Total: | 100.00 | 1002.00 | | |

The above Toner formulation was manufactured as follows:
Ingredient 1 was added into the beaker.
Ingredients 2-9 (not listed) were very slowly added into the beaker, while mixing vigorously at 250-1000 rpm using 3″ propeller blade until homogeneous.
pH was measured. The pH was adjusted to 6.0±0.25, if necessary (using either 5N HCl or 5N NaOH).

### Example 20: Dermatological Serum Composition

TABLE 20.1
An anti-aging serum was prepared according
to the formula below:

| S. No. | Phase | Ingredient | Amount (% w/w) |
|---|---|---|---|
| 1 | A | Water (aqua) | 42.35 |
| 2 | A | Ascorbic acid | 10.00 |
| 3 | B | Glutathione | 0.30 |
| 4 | C | Bis-PEG-12 Dimethicone | 1.00 |
| 5 | C | SD alcohol 40-B | 5.00 |
| 6 | D | Propanediol | 30.00 |
| 7 | D | Phenoxyethanol, | 0.75 |
| | | ethylhexylglycerin | |
| 8 | E | Ascorbyl glucoside | 16.65 |
| 9 | F | Citric acid USP 50% soln | 0.00 |
| 10 | F | Sodium hydroxide 20% Solution | 0.60 |
| | | Total | 100.0000 |

MANUFACTURING of this composition was as follows:
PHASE A Slowly add ascorbic acid to water and mix until dissolved. Warm to 35-40° C. if necessary to help with dissolution.
PHASE B Add Phase B and mix until it dissolves.
PHASE C Add Phase C ingredients one at a time to batch and mix until Bis-PEG-12 Dimethicone dissolves.
PHASE D Mix Phase D and add to batch slowly.
PHASE E Add Phase E slowly and mix until uniform.
PHASE F Adjust pH to 2.5-2.9 with Phase F if necessary.
pH: 2.59; viscosity (cps): 10 cps (water thin liquid).

### Spindle #1 @10 RPM, 1 MIN.

### Example 21: Dermatological Serum Composition

TABLE 21.1
Another anti-aging serum composition was prepared according to
the formula below (ingredients not listed in predominant order):

| S. No. | Ingredient | Amount (% w/w) |
|---|---|---|
| 1 | Water (aqua) | 71.06 |
| 2 | Ascorbic acid | 10.00 |
| 3 | Ascorbyl | 10.00 |
| | glucoside (AA- | |
| | 2G) | |
| 4 | Propanediol | 3.00 |
| 5 | Sodium hyaluronate, | 3.00 |
| | water, phenoxyethanol | |
| | mixture | |
| 6 | Sodium hydroxide | 1.59 |
| | 20% Solution | |
| 7 | Phenoxyethanol, | 0.75 |
| | ethylhexylglycerin | |
| | mixture | |
| 8 | Xanthan gum | 0.30 |
| 9 | Glutathione | 0.30 |
| | Total | 100.0000 |

### Example 22: Dermatological Serum Composition

TABLE 22.1
Another anti-aging serum composition was prepared according to the
formula below (ingredients not listed in predominant order):
pH: ~6.0.

| S. No. | Ingredient | Amount (% w/w) |
|---|---|---|
| 1 | Water (aqua) | 71.06 |
| 2 | Ascorbic acid | 10.00 |
| 3 | Ascorbyl | 10.00 |
| | glucoside | |
| 4 | Propanediol | 3.00 |
| 5 | Sodium hyaluronate, | 3.00 |
| | water, | |
| | phenoxyethanol | |
| | mixture | |
| 6 | Sodium hydroxide | 1.59 |
| | 20% Solution | |
| 7 | Phenoxyethanol, | 0.75 |
| | ethylhexylglycerin | |
| | n mixture | |
| 8 | Hydroxyethylcellulose | 0.30 |
| 9 | Glutathione | 0.30 |
| | Total | 100.0000 |

### Example 23: Dermatological Serum Composition

TABLE 23.1
Another anti-aging serum composition was prepared according to the
formula below (ingredients not listed in predominant order).
pH: ~6.0.

| S. No. | Ingredient | Amount (% w/w) |
|---|---|---|
| 1 | Water (aqua) | 63.11 |
| 2 | Ascorbic acid | 10.00 |
| 3 | Ascorbyl glucoside | 10.00 |
| 4 | SD alcohol 40-B | 5.00 |
| 5 | Propanediol | 4.00 |
| 6 | Sodium hyaluronate, | 3.00 |
| | water, phenoxyethanol | |
| | mixture | |
| 7 | Bis-PEG-12 Dimethicone | 2.00 |
| 8 | Sodium hydroxide | 1.59 |
| | 20% Solution | |
| 9 | Phenoxyethanol, | 0.75 |
| | ethylhexylglycerin | |
| | mixture | |
| 10 | Glutathione | 0.30 |
| 11 | Xanthan Gum | 0.25 |
| | Total | 100.0000 |

### Example 24: Dermatological Serum Composition

TABLE 24.1
Another anti-aging serum was prepared according to the formula
below (ingredients not listed in predominant order): pH: ~6.0.

| S. No. | Ingredient | Amount (% w/w) |
|---|---|---|
| 1 | Water (aqua) | 42.65 |
| 2 | Propanediol | 30.00 |
| 3 | Ascorbic acid | 10.00 |
| 4 | Ascorbyl glucoside | 10.00 |
| 5 | SD alcohol 40-B | 5.00 |
| 6 | Bis-PEG-12 Dimethicone | 1.00 |
| 7 | Phenoxyethanol, | 0.75 |
| | ethylhexylglycerin mixture | |
| 8 | Glutathione | 0.30 |
| 9 | Sodium hydroxide 20% Solution | 0.30 |
| | Total | 100.0000 |

The foregoing specification teaches the principles of the present invention, with description of the preferred embodiments, and with working examples provided for the purpose of illustration, so as to enable any person skilled in the art to make and use the present invention. The various modifications to these embodiments will be readily apparent to those skilled in the art, and the generic principles defined herein may be applied to other embodiments without the use of the inventive faculty. Thus, the present invention is not intended to be limited to the embodiments shown herein but is to be accorded the widest scope consistent with the principles and novel features disclosed herein and the following claims and its equivalents.

1. A dermatological cream composition comprising reduced glutathione and/or cysteinylglycine, and an antioxidant in a dermatologically acceptable carrier.

2. The dermatological cream composition of claim 1, wherein the antioxidant is at least one selected from the group consisting of ascorbyl-2-glucoside (AA-2G), ascorbyl-2-glucosamine and ascorbic acid.

3. The dermatological cream composition of claim 2, wherein said at least one antioxidant is an additional antioxidant selected from the group consisting of: carnosine, resveratrol, ascorbic acid, ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate and quercetin.

4. (canceled)

5. The dermatological cream composition according to any of claim 1 wherein the composition further comprises L-arginine.

6-14. (canceled)

15. A method for promoting healthy skin comprising applying to the skin a dermatological cream composition comprised of reduced glutathione and an antioxidant in a dermatologically acceptable carrier, wherein the dermatological cream composition is ionically balanced.

16. The method of claim 15, wherein the antioxidant is at least one of ascorbyl-2-glucoside or ascorbyl-2-glucosamine.

17. The method of claim 16 wherein the dermatological cream composition comprises ascorbic acid in addition to ascorbyl-2-glucoside or ascorbyl-2-glucosamine or in place of ascorbyl-2-glucoside and/or ascorbyl-2-glucosamine, and optionally comprises ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate and quercetin, or a combination thereof.

18. The dermatological cream composition of claim 17 wherein the composition further comprises L-arginine.

19-27. (canceled)

28. A dermatological serum composition comprising an antioxidant and reduced glutathione and/or cysteinylglycine in a dermatologically acceptable carrier.

29. The dermatological serum composition of claim 28 wherein the antioxidant is ascorbyl-2-glucoside (AA-2G) or ascorbyl-2-glucosamine.

30. The dermatological serum composition of claim 29 further comprises ascorbic acid.

31. The dermatological serum composition of claim 29, wherein the antioxidant is an additional antioxidant, or is one other than ascorbyl-2-glucoside (AA-2G) and/or ascorbyl-2-glucosamine, selected from the group consisting of: carnosine, resveratrol, ascorbic acid, ascorbyl palmitate, sodium ascorbyl phosphate, potassium ascorbyl phosphate, magnesium ascorbyl phosphate and calcium ascorbyl phosphate, ascorbyl-2-glucosamine, ascorbyl tetraisopalmitate, erythorbic acid, potassium ascorbate, sodium ascorbate, magnesium ascorbate, zinc ascorbate, molybdenum ascorbate, chromium ascorbate, manganese ascorbate, calcium ascorbate and quercetin.

32. The dermatological serum composition of claim 31 wherein the antioxidant is ascorbic acid.

33-37. (canceled)

38. A dermatological serum composition comprised of a dermatologically acceptable carrier containing reduced glutathione, ascorbyl glucoside and ascorbic acid.

39. (canceled)

40. (canceled)

41. A method for promoting healthy skin comprising applying to the skin a dermatological serum composition comprised of reduced glutathione, ascorbyl glucoside and ascorbic acid, and optionally citric acid, in a dermatologically acceptable carrier.

42. (canceled)

43. (canceled)

44. A dermatological toner composition comprised of at least 85% by weight of water based on total weight of the composition and an effective amount of sodium, magnesium, potassium, calcium and chloride ions.

45. (canceled)

46. (canceled)

47. The dermatological toner composition of claim 44, wherein said composition is ionically balanced.

48. The dermatological toner composition of claim 44, wherein pH of said composition is about 6.0.

49. The dermatological toner composition of claim 44, wherein density of said composition is about 1.0 g/ml.

50. A dermatological toner composition comprising at least 85% by weight of water based on total weight of the composition and sodium, magnesium, potassium, calcium and chloride ions, and optionally HCO₃⁻, and a preservative system comprising caprylyl glycol, wherein the dermatological toner composition is ionically balanced.

51. (canceled)
